(19) United States
(12) Patent Application Publication
(10) Pub. No.: US 2011/0117100 A1
(43) Pub. Date: May 19, 2011

(54) COMPOSITIONS AND METHODS FOR DIAGNOSIS, STRATIFICATION AND TREATMENT OF AMYLOIDOGENIC DISEASES

(75) Inventors: Markus Britschgi, Allschwil (CH); Anton Wyss-Coray, Stanford, CA (US)

(73) Assignees: The Board of Trustees of the Leland Stanford Junior University, Palo Alto, CA (US); The U.S. Government represented by the Department of Veterans Affairs, Washington, DC (US)

(21) Appl. No.: 12/611,664

(22) Filed: Nov. 3, 2009

Related U.S. Application Data
(60) Provisional application No. 61/110,761, filed on Nov. 3, 2008.
(60) Provisional application No. 61/111,180, filed on Nov. 4, 2008.

(51) Int. Cl. A61K 39/395; A61K 39/00; C07K 16/18; A61P 25/28; G01N 33/566

(52) U.S. Cl. 424/139.1; 424/185.1; 530/389.1; 530/388.9; 530/387.3; 436/501

(57) ABSTRACT

Natural human autoantibodies against toxic assemblies of Aβ and related peptide species offer a unique immune repertoire. Based on this repertoire, the present invention provides compositions and methods for diagnosis as well as for prophylactic and therapeutic treatment of neurodegenerative diseases such as Alzheimer's disease that are characterized by accumulation of beta-amyloid deposits. The same human autoantibodies may be useful for prophylactic and therapeutic treatment of other neurodegenerative disorders and systemic pathological conditions that are due to or are associated with toxic assemblies of other amyloidogenic peptides or proteins.

### RELATED APPLICATION

This application claims priority and other benefits from U.S. Provisional Patent Application Ser. 61/110,761, filed Nov. 3, 2008, entitled “Methods for Diagnosis, stratification and treatment of amyloidogenic diseases”, and No. 61/111,180, filed Nov. 4, 2008, entitled “Compositions and methods for diagnosis, stratification and treatment of amyloidogenic diseases”. The entire content of both applications is specifically incorporated herein by reference.

### STATEMENT OF GOVERNMENTAL SUPPORT

This invention was made with U.S. Government support under National Cancer Institute Grant RO1 AG020603. The Government has certain rights in this invention.

### INCORPORATION OF SEQUENCE LISTING

A computer readable form of the sequence listing, “S07_{—}339_sequences_ST25.txt”, submitted via EFS-WEB, is herein incorporated by reference.

### TECHNICAL FIELD OF THE INVENTION

The present invention relates to methods and compositions for diagnosing, stratifying and treating diseases, such as Alzheimer's Disease, that are characterized by involvement of Aβ assemblies.

### BACKGROUND

Age-related neurodegenerative illnesses, such as Alzheimer's disease (AD), vascular dementia, and Parkinson's Disease, are becoming an increasing social and economical burden as the number of older individuals continues to grow in industrialized countries. AD is still incurable and current knowledge of the debilitating processes leading to these diseases is still limited; moreover, no treatment exists that effectively prevents AD or reverses its symptoms and course.

AD is a devastating, degenerative disorder of the brain and the leading cause of dementia in the elderly, that phenotypically starts with memory loss and eventually results in complete loss of intellectual and everyday life skills Deposits of various β-amyloid peptides (Aβ) in the form of extracellular plaques and the generation of neurofibrillary tangles in the human brain are the most prevalent histopathological hallmarks of AD. Aβ is generated by proteolytic processing of the amyloid precursor protein (APP) (Haass & Selkoe, 2007) and is rapidly turned over in the healthy human central nervous system (CNS) (Bateman et al., 2006). It is speculated that the formation of Aβ plaques is due to an imbalance between Aβ production and clearance (Thal et al., 2006).

The senile plaques in AD contain mainly Aβ1-42, the 42-amino acid form of Aβ, and post-translationally modified and truncated Aβ, such as the pyroglutamate (pE) fragment Aβ3(pE)-42, and can also be detected in a large number of cognitively normal individuals (Mori et al., 1992; Naslund et al., 1994; Saido et al., 1995; Piccini et al., 2005). Aβ plaques may be in equilibrium with soluble oligomeric Aβ assemblies, which are thought to adversely affect synaptic structure and plasticity leading to neuronal death rather than the plaques themselves (Haass & Selkoe, 2007).

Removal of Aβ may reduce its toxic effects and receptor-mediated transport across the blood-brain barrier has been proposed as a potential clearance mechanism (Deane and Zlokovic, 2007). In fact, cerebral Aβ appears to be in a complex and dynamic equilibrium with plasma Aβ (Levites et al., 2006) opening the possibility that CNS Aβ levels could be reduced by trapping the peptide in a “peripheral sink” (DeMattos et al., 2001).

Immunotherapy against Aβ is one of several treatment strategies now being studied preclinically and clinically as part of efforts to discover safe and more effective means of treating Alzheimer's disease. Several effects of immunotherapies for AD are being discussed: antibody-mediated phagocytosis of Aβ, antibody-mediated shift of the equilibrium toward Aβ monomers favoring Aβ clearance and degradation, neutralization of toxic oligomeric Aβ species, peripheral Aβ sink and efflux of soluble Aβ species from the brain as well as antibody-independent, cell-mediated clearance of β-amyloid plaques (Nitsch & Hock, 2008).

Stimulating the production of Aβ antibodies by active immunization with synthetic Aβ, particularly with the beforementioned Aβ1-42, has been described with only moderate success and limited feasibility due to unwanted side effects such as aseptic meningoencephalitis (Schenk et al., 1999; Orgogozo et al., 2003; Gilman et al., 2005).

Passive immunization against AD with the administration of polyclonal or monoclonal (humanized) Aβ antibodies presents another currently investigated approach. Administering monoclonal Aβ antibodies can reduce amyloid pathology and inflammation and improve cognitive function in mouse models of AD (DeMattos et al., 2001; Levites et al., 2006; Klyubin et al., 2005.) It has, however, been found that some Aβ antibodies can enhance Aβ toxicity in vitro (Nath et al., 2003) or induce cerebral hemorrhage in APP-transgenic mice with amyloid plaques and cerebral amyloid angiopathy (Pfeifer et al., 2002).

Aβ binding autoantibodies have been detected in blood and cerebrospinal fluid (CSF) in free form or complexed with Aβ, both in AD patients and healthy individuals (Gaskin et al., 1993; Hyman et al., 2001; Nath et al., 2003; Moir et al., 2005; Jianping et al., 2006; Gruden et al., 2007, Henkel et al., 2007; Szabo et al., 2008; Gustaw et al., 2008).

The presence of such natural polyclonal Aβ autoantibodies in normal human blood has suggested the potentially therapeutic use of intravenous immunoglobulin G (IVIg) for passive immunotherapy in AD patients (Weksler et al., 2002; Dodel et al., 2002). It has also been suggested that antibodies recognizing different domains (DeMattos et al., 2001; Levites et al., 2006; Pfeifer et al., 2002; Bard et al., 2003) or conformations (Zhou et al., 2005; Mamikonyan et al., 2007) of Aβ may have different efficacy in humans.

The origin and potential physiological or pathological role of naturally occurring Aβ antibodies is, however, unclear and the literature is contradictory whether Aβ antibody titers are increased in AD (Nath et al., 2003), increased at early stages of AD (Gruden et al., 2007), correlate inversely with disease severity (Moir et al., 2005; Jianping et al., 2006; Weksler et al., 2002; Du et al., 2001) or cognitive function (Mruthinti et al., 2004) or whether levels are unchanged (Hyman et al., 2001).

Natural human autoantibodies against Aβ and related peptide species offer a unique immune repertoire, yet their uniform utilization, such as performed in IvIg treatment, is likely not as efficient as the specific usage of selected antibodies from this pool against various Aβ epitopes and assemblies.

Despite the progress which has been achieved in elucidating the underlying mechanisms of AD and related forms of dementia, there remains an urgent need to develop methods (and compositions) for treatment and early diagnosis of AD. Current diagnosis of milder forms of AD cannot reliably and easily be done by the general practitioner but has to be performed by highly trained specialists by exclusion of other neurological disorders (Dubois et al., 2007). Additionally, the above described pathological features such as neuritic plaques or neurofibrillary tangles can also be found in other forms of dementia mixed with AD and can only be assessed postmortem or, in case of plaques, can only be imaged pre-mortem at a few specialized clinical centers in the world.

### SUMMARY

In spite of the described preclinical and clinical efforts, effective, simple and safe treatment options are still not available, but urgently needed for neurodegenerative diseases such as AD. Furthermore missing are simple blood-based molecular biomarkers to assess the risk for developing AD, to diagnose the onset or progression of AD, or to monitor a potential treatment. Natural human autoantibodies against Aβ and related peptide species offer a unique immune repertoire for the diagnosis, stratification as well as treatment of neurodegenerative diseases such as AD.

Embodiments of the present invention provide methods for diagnosis as well as for prophylactic and therapeutic treatment of neurodegenerative diseases such as AD that are characterized by the involvement of Aβ assemblies.

The methods for diagnosis comprise the monitoring of specific autoantibodies to Aβ variants and assemblies for the detection of onset as well as progression of AD. The methods for stratification comprise the monitoring of specific autoantibodies to Aβ variants and assemblies for the detection of any risk to develop AD by determining any changes in expression levels of certain self antibodies that correlate with the risk of developing AD. The methods for prophylactic treatment comprise the use of identified natural antibodies for passive immunization of individuals who have been identified to be at risk for developing AD or of the general population with low levels of such natural antibodies. The methods for therapeutic treatment comprise the use of identified natural antibodies to neutralize toxic oligomeric Aβ species.

The methods for prophylactic as well as therapeutic treatment involve the use of or the production of conformation-specific antibodies, recognizing common conformation-dependent, toxic structures that are present in neurodegenerative as well as systemic amyloidopathies.

### DRAWINGS

The accompanying drawings illustrate embodiments of the invention and, together with the description, serve to explain the invention. These drawings are offered by way of illustration and not by way of limitation.

FIG. 1. Human plasma IgGs preferentially recognize oligomeric and postranslationally modified Aβ. (A) Membranes with fibrillar (f) and oligomeric (o) preparations of 2 μg Aβ1-42 were probed with biotinylated human IgG fractions isolated from plasma of two non-demented controls (NDC) and two AD patients. Bound IgGs were detected by HRP-tagged avidin. (B) In parallel experiments membranes were probed with Aβ1-5-specific mAb 3D6 (0.5 μg/ml). Since 3D6 did not detect the high molecular weight species of Aβ1-42 in the oligomeric preparation after the first probing, the lane with the fibrillar preparation was cut off and the truncated membrane strip containing oligomeric Aβ1-42 above 17 kDa was reprobed with fresh antibody. This was repeated one more time after cutting the strip above 55 kDa. (C) Fibrillar and (D) oligomeric preparations of Aβ1-42 analyzed by atomic force microscopy (5 nm total z-range). (E-G) Human IgG reactivities against peptide preparations were measured with antigen microarrays in plasma from AD patients and NDC in sample set 1 (E) and sample set 2 (F, G) (bars represent mean±SEM). Names underlined with dashed lines are synthetic mutant peptides not present in vivo. No significant differences in antibody reactivities were observed between samples from AD patients and NDC for any antigen in both sample sets (Significance Analysis of Microarray). (H, I) Titration of plasma IgG (H) and IgM (I) reactivity to oligomeric Aβ1-42 in three samples. (J, K) Antibody reactivities against groups of peptides (closed symbols) compared to reactivities against control antigens (open symbols) in sample set 2. Each dot represents median measurement for one plasma sample (log scale; mean reactivity; ***, P≦0.001; Mann-Whitney U test or Kruskal-Wallis one-way ANOVA followed by Dunn's post hoc test). (L, M) Electron microscopy analysis of assembly status of non-aggregated (L) and oligomeric preparation (M) of Bri16-23(A16K/V17K). DFU, digital fluorescent units.

FIG. 2. Reactivity to oligomeric assemblies of Aβ peptides decline with progression of AD and together with other reactivities also decrease with age. (A, B) Average antibody reactivity for Aβ1-42 oligomer preparations in AD patients with mild (MMSE score≧20, orange dots) or moderate to severe stages (MMSE score≦19, red dots) of the disease in sample set 1 (A) and sample set 2 (B) (mean reactivity; **P≦0.01, Mann Whitney U test). Each dot represents median measurement for one plasma sample. (C) Unsupervised clustering of antibody reactivities that are significantly associated with age in healthy females (n=66) ages 21-85 years (Significance Analysis of Microarray). Age of donors is indicated on the right of the node map as boxes with increasing intensities of purple for higher age. Color shades of the node map indicate higher (red) or lower (blue) antibody reactivity. (D) ELISA measurements for free (untreated) and total (immune complex dissociation at pH 3.5) antibody reactivity against oligomeric species of Aβ1-42 in randomly selected plasma samples of healthy individuals from different age groups (n=11-15 samples per group) of sample set 3 and AD patients (n=13) of sample set 2 (mean; one-way ANOVA and Tukey's post hoc test). DFU, digital fluorescent units; OD450, optical density at 450 nm.

FIG. 3. IgGs to Aβ are present in human CSF, protect neurons from Aβ toxicity in vitro, and are amplified together with ABri and ADan cross-reactive antibodies in an Aβ immunization paradigm. (A) IgG reactivity pattern in CSF of AD patients (≧70 y) and non-demented (NDC) young (24-48 y) and aged individuals (58-83 y) (bars represent means of median reactivities±SEM). (B, C) Protective effect of human IgGs on primary mouse E16 neurons incubated with oligomeric preparation of Aβ1-42 was measured by (B) percent lactate dehydrogenase (LDH) release in dying cells and by (C) percent generation of intracellular formazan in surviving cells. Amount of water-soluble formazan produced by cells in culture medium alone was considered 100% survival (bars represent mean±SEM of three experiments done in triplicates; symbols in scatter plot represent mean of one triplicate experiment; *P≦0.05, one-way ANOVA and Dunnett's multiple comparison test with ‘no antibody’ as control). (D, E) Vervet monkeys were (D) left untreated or (E) immunized with a cocktail of Aβ1-40 and Aβ1-42 (Lernere et al., 2004). Immunoreactivity at the end of the study (day 301) was divided by the baseline reactivity to individual peptides and result is presented as log fold change (bars represent median fold change of reactivity to a peptide for each individual animal). As expected, immunoreactivity to Aβ1-40 and Aβ1-42 peptides and oligomeric assemblies increase over several log ranges. Note the increase in reactivity to Aβ11(pE)-42, ABri, and ADan but the lack of increase to Aβ33-42. DFU, digital fluorescent units.

FIG. 4. Reactivity of human IgGs on peptide microarray. (A) Peptides printed in an ordered array on glass slides were probed with human plasma. Bound IgGs were detected by fluorescently tagged anti-human IgG secondary antibodies. Assembly of ten of a total of 16 blocks of a representative array is shown. Spot sizes for antigens range from 10 μm to about 150 μm in diameter. (B) Higher magnification of upper right block. Every antigen is spotted in quadruplicates and some peptides or preparations were printed repeatedly.

FIG. 5. Titer measurements of reactivity to Aβ3(pE)-42 and increased reactivity of posttranslationally modified peptides. (A, B) Titration of plasma IgG (A) and IgM (B) reactivity to Aβ3(pE)-42 in three samples. (C, D) Antibody reactivities against synthetic peptides of pyroglutamate modified (C) Bril-6 or (D) oxidized (Ox) Aβ1-42 (both closed symbols) compared to reactivities against control antigens (open symbols) in sample set 2 (mean reactivity; ***, P≦0.001; ****, P≦0.0001; Mann-Whitney U test). Each dot represents median measurement for one plasma sample. DFU, digital fluorescent units.

FIG. 6. Baseline plasma antibody reactivity in healthy females against various synthetic Aβ species and non-Aβ peptides. (A, B) Antibody reactivities against synthetic peptide preparations were measured with antigen microarrays in plasma from healthy females of sample set 3 (bars represent mean±SEM). Names underlined with dashed line are synthetic mutant peptides not present in vivo. DFU, digital fluorescent units.

FIG. 7. Antibody reactivities against oligomeric, non-aggregated, and certain mutant Aβ decrease in healthy individuals with age. (A-F) All antibody reactivities that are significantly decreased in old (71-85 years, n=21, dark purple dots) compared to young (21-44 years, n=18, open circles) healthy females of sample set 3 (Significance Analysis of Microarray). For peptides prepared and printed repeatedly, the average antibody reactivity was taken in this analysis. (G-J) Representative high antibody reactivities that remain unchanged in old females compared to young (mean; 0% FDR considered significant in two-class multiple comparison analysis in SAM; P, Mann-Whitney U test post hoc). (K) Unchanged total IgG levels in healthy old females compared to young (Mann-Whitney U test). (L, M) Reactivity to positive control (L) C. albicans and does not change with age whereas reactivity to (M) pneumococcal vaccine increases slightly indicating that there is not a general decrease of immunoreactivity with age. (N, O) Scatter plots of age versus antibody reactivity against the two antigens that were found to have strongest association with age in healthy females (n=66) (linear regression modeling of age with antibody reactivities done with elastic net; rₛ, Spearman rank correlation coefficient; trend line with 95% confidence interval calculated post hoc for illustration). DFU, digital fluorescent units.

FIG. 8. Percentage free Aβ1-42 oligomer antibodies in plasma of healthy controls and AD patients. Total (immune complex dissociation at pH 3.5) and free (untreated) antibody reactivity against oligomeric species of Aβ1-42 was measured by ELISA in randomly selected plasma samples of healthy individuals from different age groups and AD patients. Measurement for total reactivity (see FIG. 2) was considered 100% and average percentage free of total reactivity was calculated.

FIG. 9. Antibody reactivities in immunized and control vervet monkeys at baseline at the end of the study. (A-D) Immunoreactivities in plasma of vervets at begin (baseline) (A, C) and at the end of the study (day 301) (B, D). Vervets 1-3 were left untreated (A, B) and vervets 4-6 were immunized (C, D) with a cocktail of Aβ1-40 and Aβ1-42 (Lernere et al., 2004). Bars represent median immunoreactivity to a peptide for each individual animal. Note the log scale. DFU, digital fluorescent units.

### TABLES

TABLE 1
Peptides and proteins on microarray

| Antigens^{a} | Source^{b} | Array^{c} |
|---|---|---|
| Aβ1-40_1^{d} | JG | 1, 2, 3 |
| Aβ1-40_2 | Ba | 1, 2 |
| Aβ1-40_3 | YU | 1, 2 |
| Aβ1-42_1 | YU | 1, 2 |
| Aβ1-42_2 | YU | 1, 2 |
| Aβ1-42_3 | Ba | 1 |
| Aβ1-42_4 | rP | 2 |
| Aβ1-42_5 | rP | 2 |
| Aβ1-42_6 | JR | 2 |
| Aβ1-42_7 | rP | 3 |
| Aβ1-40 dimer^{d} | JG | 2 |
| Aβ1-40 polymer^{d} | JG | 2 |
| Aβ1-42 polymer^{d} | JG | 2 |
| Aβ1-40 oligo 1 | Ba | 1 |
| Aβ1-40 oligo 2 | Ba | 1 |
| Aβ1-42 oligo 1 | Ba | 1 |
| Aβ1-42 oligo 2 | Ba | 1 |
| Aβ1-42 oligo 3 | rP | 2 |
| Aβ1-42 oligo 4 | JR | 2 |
| Aβ1-42 oligo 5 | JR | 2 |
| Aβ1-42 oligo 6 | rP | 3 |
| Aβ1-42 oligo 7 | rP | 3 |
| Aβ1-42 oligo 8 | rP | 3 |
| Aβ1-42 CAPS 1 | Ba | 1, 3 |
| Aβ1-42 CAPS 2 | rP | 2 |
| Aβ1-42 CAPS 3 | rP | 2 |
| Aβ1-42 CAPS 4 | JR | 2 |
| Aβ1-42 CAPS 5 | JR | 2 |
| Aβ1-42 CAPS 6 | JR | 2 |
| Aβ1-40 fibril | Ba | 1 |
| Aβ1-42 fibril 1 | Ba | 1 |
| Aβ1-42 fibril 2 | Ba | 1 |
| Aβ1-42 fibril 3 | JR | 2 |
| Aβ1-42 fibril 4 | JR | 2 |
| Aβ10-20 | Ba | 2 |
| Aβ11-42 | YU | 2 |
| Aβ22-35 | YU | 2 |
| Aβ33-42 | YU | 2, 3 |
| Aβ1-42 (Ox) | YU | 2 |
| Aβ29-35(MetSox) | YU | 2 |
| Aβ3(pE)-40 | YU | 1, 2 |
| Aβ11(pE)-40 | YU | 1, 2 |
| Aβ3(pE)-42 | YU | 2, 3 |
| Aβ11(pE)-42 | YU | 2, 3 |
| Aβ1-40 (D7N) Tottori | YU | 1, 2 |
| Japanese | | |
| Aβ1-40 (D23N) Iowa_1 | YU | 1, 2 |
| Aβ1-40 (D23N) Iowa_2 | YU | 2 |
| Aβ1-40 (A21G) Flemish | YU | 2 |
| Aβ1-40 (E22Q) Dutch_l | YU | 1, 2 |
| Aβ1-40 (E22Q) Dutch_2 | YU | 1, 2 |
| Aβ1-40 (E22Q) Dutch_3 | YU | 2 |
| Aβ1-42 (E22Q) Dutch | YU | 2, 3 |
| Aβ1-42 (E22G) Arctic | YU | 1, 2, 3 |
| Aβ1-42 (E22K) Italian | YU | 1, 2 |
| Aβ1-40 (R5G/Y10F/H13R) | YU | 2, 3 |
| rodent | | |
| Aβ1-42 (R5G/Y10F/H13R) | YU | 2, 3 |
| rodent | | |
| Aβ1-55 | YU | 2, 3 |
| Bri1-23 | YU | 1, 2, 3 |
| Bri9-22 | YU | 2 |
| Bri13-22 | YU | 2, 3 |
| Bri1-6 | YU | 2 |
| Bri1(pE)-6 | YU | 2 |
| Bri16-23 (A16K/V17K) | YU | 1, 2 |
| Bri16-23 (A16K/V17K) oligo | YU | 2 |
| ABri1(pE)-34 | YU | 2, 3 |
| ABri3-34 | YU | 1, 2, 3 |
| ABri22-34 | YU | 2, 3 |
| ABri24-34 | YU | 2, 3 |
| ABri3-34 oligo | YU | 2 |
| ABri1(pE)-34 (Ox) | YU | 2 |
| ABri1(pE)-34 (C22S) | YU | 2 |
| ABri29-34 (N29C) | YU | 2 |
| ABri1(pE)-34 (C5S)_1 | YU | 2 |
| ABri1(pE)-34 (C5S)_2 | YU | 2 |
| ABri1(pE)-34 (C5S)_2 oligo | YU | 2 |
| ADan1(pE)-25 | YU | 2 |
| ADan1(pE)-25 SS cyclized | YU | 2 |
| ADan1(pE)-34 | YU | 2, 3 |
| ADan22-34 | YU | 2 |
| APLP1 (559-580) | YU | 2, 3 |
| APLP2 (662-686 | YU | 2 |
| Amylin1-37 | YU | 2, 3 |

| Controls and markers | |
|---|---|
| Candida albicans extract | 2, 3 |
| Pneumococcal vaccine, polyvalent | 1, 2, 3 |
| anti-human IgG/IgM | 1, 2 |
| anti-human IgG (H + L) | 1, 2 |
| BSA-Cy3 | 1, 2 |

^{a}Single letter amino acid peptide sequence, in vivo precursor protein nomenclature, and Swiss-Prot accession number:
Aβ1-42: DAEFRHDSGYEVHHQKLVFFAEDVGSNKGAIIGLMVGGVVIA, APP (672-713), P05067
Aβ1-55: DAEFRHDSGYEVHHQKLVFFAEDVGSNKGAIIGLMVGGVVIATVIVITLVMLKKK, APP (672-726), P05067
Bri1-23: EASNCFAIRHFENKFAVETLICS, BRI2 (244-266), Q9Y287
ABri1-34: EASNCFAIRHFENKFAVETLICSRTVKKNIIEEN, ABriPP (244-277), Q9Y287
ADan1-34: EASNCFAIRHFENKFAVETLICFNLFLNSQEKHY, ADanPP (244-277), Q9Y287
Amylin1-37: KCNTATCATQRLANFLVHSSNNFGAILSSTNVGSNTY, IAPP (34-70), P10997
APLP1 (559-580): PFHSSDIQRDELARSGTGVSRE, P51693
APLP2 (662-686): KEMIFNAERVGGLEERESGPRLE, Q06481
^{b}Ba, Bachem; JG, Jorge Ghiso; JR, Jayakumar Rajadas; rP, rPeptide; YU, Yale University
^{c}1, printed for human sample set 1; 2, printed for human sample sets 2 and 3; 3, printed for human plasma titration, human CSF and vervet samples.
^{d}separated by HPLC
Mutations of human Aβ1-40 or Aβ1-42 are listed together with their commonly used names. Rodent Aβs differ from human Aβ by the three indicated amino acids. Additional reference about posttranslationally modified Aβ peptides or assemblies of Aβ that were isolated from the healthy human brain or from brains of patients with AD (10-12).
Human natural antibodies have been reported only against a few of the listed Aβ peptides and assemblies, e.g. for Aβ1-40 or Aβ1-42 (13-23), Aβ25-35 (15, 24) (not on our array but similar to Aβ22-35, which is on the array), Aβ1-x oligomers (although the assembly structure was not confirmed (7)), Aβ1-42 CAPS (4), or fibrillar Aβ assemblies (25). Natural antibodies have also been described to react with plaques in brain sections of AD patients (13, 20) and AD mouse models (26) which contain various Aβ assemblies including fibrils and posttranslationally modified and truncated Aβ amongst many other proteins. Other reactivities to printed peptides reported by us seem to be novel.
Aβ, β-amyloid; ABri, amyloid British peptide; ABriPP, ABri precursor protein; ADan, amyloid Danish peptide; ADanPP, ADan precursor protein; APLP, amyloid-β (A4) precursor protein-like protein; Bri, BRI2 peptide; BSA, bovine serum albumin; fibril, fibrillar preparation; CAPS, dityrosine cross-linked Aβ peptide species; IAPP, islet amyloid polypeptide precursor, MetSox, methionine sulfoxide; oligo, oligomeric preparation; Ox, oxidized; pE, pyroglutamate.

TABLE 2
Subject demographic characteristics

| | | | | |
|---|---|---|---|---|
| | | Age | | MMSE score |
| Sample | | mean ± SD | Sex | mean ± SD |
| description | Number | (range) | % female | (range) |

Plasma samples 1 ^{a}

| | | | | |
|---|---|---|---|---|
| Non-demented | 36 | 73.1 ± 6.1 | 54.3% | 29.1 ± 1.2 |
| controls (NDC) | 75 | (65-86) | 69.1% | (26-30) |
| Alzheimer's | | 75.7 ± 7.9 | | 13.8 ± 8.0 |
| disease (AD) | | (46-96) | | (0-28) |

Plasma samples 2 ^{b}

| | | | | |
|---|---|---|---|---|
| Non-demented | 62 | 71.8 ± 10.1 | 83.9% | 29.1 ± 1.0 |
| controls (NDC) | 55 | (51-89) | 81.8% | (26-30) |
| Alzheimer's | | 73.0 ± 9.2 | | 22.8 ± 5.5 |
| disease (AD) | | (51-91) | | (4-30) |

Plasma samples 3 ^{c}

| | | | | |
|---|---|---|---|---|
| Healthy females | 66 | 56.0 ± 19.8 | 100.0% | 29.5 ± 0.6 |
| | | (21-85) | | (28-30) |

CSF samples ^{b,c}

| | | | | |
|---|---|---|---|---|
| Non-demented | 16 | 56.8 ± 19.7 | 100.0% | 29.5 ± 0.7 |
| controls (NDC) | 6 | (24-83) | 100.0% | (28-30) |
| Alzheimer's | | 76.2 ± 5.5 | | 26.2 ± 2.1 |
| disease (AD) | | (70-84) | | (22-28) |

Vervet plasma samples ^{d}

| | | | | |
|---|---|---|---|---|
| Control vervets | 3 | 24.7 ± 4.7 | 100.0% | n.a. |
| Immunized | 3 | (21-30) | 66.7% | n.a. |
| vervets | | 18.7 ± 3.1 | | |
| | | (16-22) | | |

^{a} University of Wroclaw and Stanford University/VA Palo Alto. Information on age was obtained for 66 AD and 35 NDC and on MMSE for 70 AD and 27 NDC.
^{b} VA Puget Sound Health Care System, UCSD, Oregon Health Sciences University, University of Pennsylvania, and Washington University.
^{c} Samples were collected for a healthy aging study at VA Puget Sound Health Care System, UCSD, University of Oregon, and University of Pennsylvania. Some 42 samples from older female donors were part of NDC in plasma sample set 2.
^{d}Lemere C A et al. (2004)
MMSE, Mini Mental State Exam (Folstein et al., 1975);
SD, standard deviation;
n.a., not applicable.

TABLE 3
Selected peptides and proteins on microarray

| Antigens | Antigens |
|---|---|
| Aβl-40 dimer | Bri1(pE)-6 |
| Aβ1-40 polymer | Bri16-23 (A16K/V17K) |
| Aβ1-42 polymer | Bri16-23 (A16K/V17K) oligo |
| Aβ1-40 oligo 1 | ABri1(pE)-34 |
| Aβ1-40 oligo 2 | ABri3-34 |
| Aβ1-42 oligo 1 | ABri22-34 |
| Aβ1-42 oligo 2 | ABri24-34 |
| Aβ1-42 oligo 3 | ABri3-34 oligo |
| Aβ1-42 oligo 4 | ABri1(pE)-34 (Ox) |
| Aβ1-42 oligo 5 | ABri1(pE)-34 (C22S) |
| Aβ1-42 CAPS 1 | ABri29-34 (N29C) |
| Aβ1-42 CAPS 2 | ABri1(pE)-34 (C5S)_2 |
| Aβ1-42 CAPS 3 | ABri1(pE)-34 (C5S)_2 oligo |
| Aβ1-42 CAPS 4 | ADan1(pE)-25 |
| Aβ1-42 CAPS 5 | ADan1(pE)-34 |
| Aβ1-42 CAPS 6 | ADan22-34 |
| Aβ10-20 | APLP1 (559-580) |
| Aβ1-42 (Ox) | APLP2 (662-686 |
| Aβ29-35(MetSox) | Amylin1-37 |
| Aβ3(pE)-40 | |
| Aβ11(pE)-40 | |
| Aβ3(pE)-42 | |
| Aβ11(pE)-42 | |
| Aβ1-40 (E22Q) Dutch_1 | |
| Aβ1-40 (E22Q) Dutch_2 | |
| Aβ1-40 (E22Q) Dutch_3 | |
| Aβ1-42 (22Q) Dutch | |
| Aβ1-42 (E22G) Arctic | |
| Aβ1-42 (E22K) Italian | |

### DEFINITIONS

Unless defined otherwise, all technical and scientific terms used herein have the same meaning as commonly understood by one of ordinary skill in the art to which this invention belongs. Sambrook and Russell (2001) Molecular Cloning: A Laboratory Manual (Third Edition), Cold Spring Harbor Laboratory Press, Cold Spring Harbor, N.Y., is a good reference for definitions, terms of art and standard methods known in the art. Although any methods and materials similar or equivalent to those described herein can also be used in the practice or testing of the present invention, representative illustrative methods and materials are herein described.

“Amyloidogenic disease” or “amyloidosis” refers to any disorder which is characterized by an accumulation or formation of amyloid plaques as a symptom or as part of its pathology.

An “amyloid plaque” is an extracellular deposit composed mainly of proteinaceous fibrils. Generally, the fibrils are composed of a dominant protein or peptide.

The term “peptide” also refers to a compound composed of amino acid residues linked by peptide bonds. Generally peptides are composed of 100 or fewer amino acids, while polypeptides or proteins have more than 100 amino acids. As used herein, the term “protein fragment” may also be read to mean a peptide.

An “epitope” is the part of an antigen that binds to the antigen-binding region of an antibody.

The terms “Aβ,” “Aβ peptide” and “Amyloid β” peptide are synonymous, and refer to one or more peptide compositions of about 38-55 [comment MBr: e.g. Aβ1-55] amino acids derived from beta amyloid precursor protein (β-APP).

A “pharmaceutical composition” refers to a chemical or biological composition suitable for administration to a mammalian individual. Such compositions may be specifically formulated for administration via one or more of a number of routes, including but not limited to, oral, parenteral, intravenous, intraarterial, subcutaneous, intranasal, sublingual, intraspinal, intracerebroventricular, and the like.

The terms “natural” and “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is natural or naturally occurring.

The term “effective dose” as used herein describes a dose that is able to elicit a quantifiable immune response.

### DETAILED DESCRIPTION

Embodiments of the present invention provide compositions and methods for diagnosis as well as for prophylactic and therapeutic treatment of neurodegenerative, amyloidogenic diseases including Alzheimer's disease that are characterized by accumulation of beta-amyloid (Aβ) peptides or related amyloidogenic peptides or proteins. Amyloidogenic diseases encompass pathological conditions that are characterized by the development and/or existence of amyloid fibrils which can be intracellular or extracellular protein deposits. Amyloidogenic diseases are typically localized, but can occur in systemic form as well.

Localized amyloidogenic diseases generally only affect a single organ and are usually characterized by specific protein deposits.

In one aspect, the present invention provides methods to treat a subject suffering from neurodegenerative conditions including beta amyloid-related diseases such as Alzheimer's disease with an effective dose of a peptide that is a higher order assembly of Aβ peptides, oxidized Aβ, pyroglutamate Aβ and selected Bri, ABri or ADan peptides and their variants to elicit active immunization.

In another aspect, the present invention provides methods to treat a subject suffering from neurodegenerative conditions including beta amyloid-related diseases such as Alzheimer's disease with an effective dose of an antibody against a peptide that is a higher order assembly of Aβ peptides, oxidized Aβ, pyroglutamate Aβ and selected Bri, ABri or ADan peptides and their variants to elicit passive immunization.

In yet another aspect, the present invention provides methods of assessing the risk of a subject to develop a beta amyloid-related disease including Alzheimer's disease by quantifying the expression of a natural antibody specific for a peptide that is a higher order assembly of Aβ peptides, oxidized Aβ, pyroglutamate Aβ and selected Bri, ABri or ADan peptides and their variants.

In another aspect, the present invention provides methods of diagnosing a subject with a beta amyloid-related disease including Alzheimer's disease detecting a natural antibody specific for a peptide that is a higher order assembly of Aβ peptides, oxidized Aβ, pyroglutamate Aβ and selected Bri, ABri or ADan peptides and their variants.

Embodiments of the invention also include the use of humanized antibodies, human antibodies secreted by immortalized human B cells or hybridomas for treating, diagnosing, preventing or reversing Alzheimer's disease or other amyloidopathies or to inhibit the formation of amyloid plaques or the effects of toxic soluble Aβ or other amyloidogenic peptide or protein species in humans.

In a further aspect of the present invention, the antibodies according to the invention are used in conventional immunological techniques for the detection of Aβ peptides wherever they may occur, including biological samples for the monitoring of beta amyloid-related diseases. Suitable immunological techniques are well known to those skilled in the art and include, for example, ELISA, Western Blot analysis, competitive or sandwich immunoassays and the like, utilizing the formation of an antigen-antibody immune complex wherein, for the purpose of the assay, the antibody can be detectably labeled with a radio, enzyme, fluorescent or other label or it can be immobilized on insoluble carriers.

Subjects amenable to treatment and diagnosis, as described by the various aspects of the present invention, include individuals at risk of disease, but not showing symptoms as well as patients presently showing symptoms. In the case of Alzheimer's disease, everyone is at risk of suffering from Alzheimer's disease eventually, whereby the risk directly correlates with advanced age. The present methods are also useful for individuals who have a known genetic risk of developing Alzheimer's disease. It is perceivable that the present methods can be administered prophylactically from a certain age on with or without preceeding assessment of the risk of developing an amyloidogenic disease. However, the stratification of a subject, e.g., the assessment of the subject's risk to develop an amyloidogenic disease using the present methods might be helpful in determining the appropriate age at which a prophylactic treatment should be started.

Such individuals include those having relatives who have experienced this disease, and those whose risk is determined by analysis of genetic or biochemical markers. Genetic markers of risk toward Alzheimer's disease include certain mutations in the APP gene, that cause increased amyloidogenicity in the Aβ peptide. Other markers of risk are mutations in the presenilin genes, PS1 and PS2, the ApoE4 genotype or family history of AD. Individuals suffering from Alzheimer's disease can be recognized by gradually debilitating dementia according to the NINCDS-ADRDA Alzheimer's Criteria (Dubois et al., 2007).

In a prophylactic treatment regimen, effective doses of the peptides (for active immunization) or their antibodies (for passive immunization) of the present invention may be administered to a subject who has or has not been found at risk for developing an amyloidogenic disease in an amount sufficient to reduce the risk or delay the onset of the disease.

In a therapeutic treatment regimen, effective doses of the peptides (for active immunization) or their antibodies (for passive immunization) of the present invention may be administered to a subject who is already suffering from an amyloidogenic disease in an amount sufficient to bring the disease to a halt, and/or dissolve the amyloid plaques and/or block the formation of new amyloid plaques and/or toxic soluble non-fibrillar, oligomeric assemblies.

Effective doses of the compositions (peptides and antibodies) of the present invention, for the above described prophylactic or therapeutic treatment regimens, may vary, since they depend upon many different factors, including means of administration, target site, physiological state of the patient, additional use of adjuvant, whether the patient is human or non-human, whether other medications are co-administered, and whether treatment is prophylactic or therapeutic. Furthermore, the dosage and frequency of repeated administration to achieve the desired prophylactic or therapeutic effect will depend on the described different factors; some subjects may receive treatment all their live long.

Effective doses of the compositions (peptides and antibodies) of the present invention can be given by parenteral, topical, intravenous, oral, subcutaneous, intraperitoneal, intranasal or intramuscular administration for prophylactic and/or therapeutic treatment.

The compositions of the present invention can be administered in combination with an adjuvant. Those adjuvants ideally augment the intrinsic response to an antigen without causing conformational changes in the antigen that could affect the quality of the response. Adjuvants can be administered as a component of a therapeutic composition together with an antigen or antibody of the present invention or can be administered separately, before, concurrently with, or after administration of the antigen or antibody of the present invention.

Methods of Diagnosis. Embodiments of the present invention also provide methods of detecting a natural antibody specific for a peptide that is a higher order assembly of Aβ peptides, oxidized Aβ, pyroglutamate Aβ and selected Bri, ABri or ADan peptides and their variants in a subject suffering from or susceptible to an amyloidogenic disease. The methods are particularly useful for monitoring a course of treatment being administered to a subject. The methods can be used to monitor both therapeutic treatment on symptomatic subjects and prophylactic treatment on asymptomatic subjects. The methods are useful for monitoring both active immunization (e.g., antibody produced in response to administration of antigen) and passive immunization (e.g., measuring level of administered antibody).

Compositions of the present invention also include antibodies and autoantibodies that specifically bind to antigens listed in Table 3 and that can be isolated from polyclonal sera by affinity purification (immunoaffinity chromography) using the individual antigens shown in Table 3. Such antibodies can be monoclonal or polyclonal.

Polyclonal sera typically contain mixed populations of antibodies binding to several epitopes along the length of Aβ. However, polyclonal sera can be specific to a particular segment of an antigen listed in Table 3. Monoclonal antibodies bind to a specific epitope within an antigen listed in Table 3 that can be a conformational or nonconformational epitope. Prophylactic and therapeutic efficacy of antibodies can be tested using transgenic animal models, e.g. human wildtype or mutant APP-transgenic mice.

Immunoglobulins or antibodies. The basic antibody structural unit is known to comprise a tetramer of subunits. Each tetramer is composed of two identical pairs of polypeptide chains, each pair having one light and one heavy chain. The amino-terminal portion of each chain includes a variable region of about 100 to 110 or more amino acids primarily responsible for antigen recognition. The carboxy-terminal portion of each chain defines a constant region primarily responsible for effector function, see generally William Paul, Fundamental Immunology, 4^{th} edition (1999), Lippincott Williams & Wilkins.

Light chains are classified as either kappa or lambda. Heavy chains are classified as gamma, mu, alpha, delta, or epsilon, and define the antibody's isotype as IgG, IgM, IgA, IgD and IgE, respectively. The variable regions of each light/heavy chain pair form the antibody binding site. Thus, an intact antibody has two binding sites. With the exception of bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4.

Production of antibodies. Apart from isolation, antibodies can be obtained by a variety of means.

Production of non-human antibodies. The production of non-human monoclonal antibodies, e.g., mouse, rat and other small animals, is well known and can be accomplished by, for example, immunizing the animal with a preparation containing an antigen from Table 3 or an immunogenic fragment of an antigen from Table 3. For those purposes, the antigen can be obtained from a natural source, by peptide synthesis or by recombinant expression. Rabbits or guinea pigs are typically used for making polyclonal antibodies. Mice are typically used for making monoclonal antibodies. Antibodies are screened for specific binding to the antigen. Often, antibodies are further screened for binding to a specific region of the antigen. Binding can be assessed, for example, by Western blot or ELISA. The smallest fragment to show specific binding to the antibody defines the epitope of the antibody. Alternatively, epitope specificity can be determined by a competition assay in which a test and reference antibody compete for binding to the component. If the test and reference antibodies compete, then they bind to the same epitope or epitopes sufficiently proximal that binding of one antibody interferes with binding of the other.

Production of humanized antibodies. Humanized antibodies have the same or similar binding specificity and affinity as a mouse or other nonhuman antibody that provides the starting material for construction of a humanized antibody. Humanized antibodies have variable region framework residues substantially from a human antibody (termed an acceptor antibody) and complementarity determining regions substantially from a mouse-antibody (termed a donor antibody). The constant region(s), if present, are also substantially or entirely from a human antibody, see William Paul, Fundamental Immunology, 4^{th} edition (1999), Lippincott Williams & Wilkins. The human variable domains are usually chosen from human antibodies whose framework sequences exhibit a high degree of sequence identity with the murine variable region domains from which the CDRs were derived. The heavy and light chain variable region framework residues can be derived from the same or different human antibody sequences. The human antibody sequences can be the sequences of naturally occurring human antibodies or can be consensus sequences of several human antibodies.

Production of human antibodies. Human antibodies against amyloid peptides can be produced from non-human transgenic mammals having transgenes encoding at least a segment of the human immunoglobulin locus. Human polyclonal antibodies can also be provided in the form of serum from humans immunized with an immunogenic agent. Such polyclonal antibodies can be concentrated by affinity purification using Aβ or another amyloid peptide as an affinity reagent.

Expression of Recombinant Antibodies. Humanized and human antibodies can be produced by recombinant expression. Recombinant constructs typically include an expression control sequence operably linked to the coding sequences of antibody chains, including naturally-associated or heterologous promoter regions. An expression control sequences is typically a eukaryotic promoter system in a vector capable of transforming or transfecting eukaryotic host cells. Once the vector has been incorporated into the appropriate host, the host is maintained under conditions suitable for high level expression of the nucleotide sequences, and the collection and purification of the crossreacting antibodies. Expression vectors are typically replicated in the host organisms either as episomes or as an integral part of the host chromosomal DNA. Expression vectors generally contain selection markers to allow detection of those cells that were transformed with the desired DNA sequences.

E. coli (prokaryotic) and Saccharomyces (eukaryotic) are useful hosts for cloning, see Ernst Winnacker, From Genes to Clones: Introduction to Gene Technology (1987), VCH Publishers, NY. A number of mammalian host cell lines capable of secreting intact heterologous proteins have been developed in the art, and include CHO cell lines, COS cell lines, HeLa cells, L cells and myeloma cell lines.

Amyloidogenic Diseases or Amyloidoses

Amyloidogenic diseases or amyloidoses include a number of disease states exhibiting various symptoms. These disorders have in common the presence of abnormal extracellular deposits of protein fibrils, known as “amyloid deposits” or “amyloid plaques” that are usually about 10-100 μm in diameter and are localized to specific organs or tissue regions. The peptides or proteins forming the plaque deposits are often produced from a larger precursor protein. More specifically, the pathogenesis of amyloid fibril deposits generally involves proteolytic cleavage of an “abnormal” precursor protein into fragments. These fragments generally aggregate into anti-parallel β pleated sheets. Local amyloid plaques occur commonly in the brain, particularly in elderly individuals over the age of 65. The most frequent type of amyloid in the brain is composed primarily of Aβ peptide fibrils, resulting in dementia.

Besides Alzheimer's disease, Parkinson's disease, Lewy body dementia, Huntington's disease, tauopathy, synucleinopathy, prion disease, and neurodegenerative diseases with TDP-43 aggregates are all characterized by the formation of toxic assemblies of amyloidogenic proteins, presumably mostly in oligomeric conformation.

Certain familial forms of Alzheimer's disease, as well as Down's syndrome, are the result of mutations in beta amyloid precursor protein, resulting in deposition of plaques having fibrils composed mainly of β-amyloid peptide (Aβ); symptoms are then observable in individuals already in their thirties or forties.

Familial British dementia (FBD) or familial Danish dementia (FDD) are characterized by progressive cognitive impairment, spasticity, and cerebellar ataxia with neurofibrillar degeneration and widespread parenchymal and vascular amyloid deposits. These forms of dementia are associated with a mutation in the BRI gene, resulting in the production of an amyloidogenic fragment, amyloid-Bri (ABri) for Familial British dementia and Adan for familial Danish dementia.

Amyloidogenic peptides, proteins, peptide or protein fragments and such can be synthesized by solid phase peptide synthesis or recombinant expression, according to standard methods well known in the art, or can be obtained from natural sources following purification chromatography. Recombinant expression can be in bacteria, such as E. coli, yeast, insect cells or mammalian cells; alternatively, proteins can be produced using cell free in vitro translation systems known in the art. Procedures for recombinant expression are described by Sambrook and Russell (2001) Molecular Cloning: A Laboratory Manual (Third Edition), Cold Spring Harbor Laboratory Press, Cold Spring Harbor, N.Y.

Monomeric beta amyloid is a peptide or posttranslationally modified fragment of Aβ with up to about 4.5 kD molecular weight (e.g. Aβ1-42). The peptides or fragments are produced synthetically or isolated from cells cultures expressing human APP or extracted from brains of humans or animal models such as APP-transgenic mice.

Multimeric assembly of beta amyloid are at least dimers and can be soluble oligomers or proto-fibrils or water insoluble high molecular weight fibrils. Such assemblies can be produced from synthetic monomeric full length Aβ or fragments of Aβ following published protocols by incubation under certain buffer conditions with or without chemical cross-linking of certain amino acids, e.g. dityrosine formation at Tyr10. Such assemblies can also be isolated from cells cultures expressing human APP or extracted from brains of humans or animal models such as APP-transgenic mice.

All publications, patent applications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

### EXPERIMENTAL

The following methods and materials were used in the examples that are described further below.

Plasma and CSF samples. A total of 252 archived human EDTA plasma samples and 22 CSF samples were obtained from academic centers specialized in neurological or neurodegenerative diseases (Table 2). Informed consent was obtained from all human subjects in accordance with IRB-approved protocols. Vervet plasma samples came from a previously published Aβ immunization study (Lernere et al., 2004). One control animal was not part of the original study. The animals were housed at the Caribbean Primate Laboratories, Behavioral Science Foundation (BSF) in St. Kitts, Eastern Caribbean, which is a fully accredited biomedical research facility with approvals from the Canadian Council for Animal Care (CCAC) and the USPHS.

Peptides, proteins, peptide aliquoting and assembly preparation. Most peptides were produced and reverse-phase HPLC purified by James Eliott (Yale University, New Haven Conn., USA) and one Aβ1-42 peptide was made at Stanford (Table 1). Other sources of Aβ1-40, Aβ1-42, or short Aβ fragments included Bachem (Torrance Calif., USA), American Peptide (Sunnyvale Calif., USA), and rPeptide (Bogart Ga., USA). Preparation of Aβ assemblies was done as described earlier (Moir et al., 2005; Stine et al., 2003). Candida albicans extract, pneumococcal vaccine, polyvalent, monoclonal antibodies recognizing human IgG (H+L) or IgG/IgM, and Cy3-labeled BSA were used as controls. Buffers and solutions for peptide assemblies were all made in HPLC grade H₂O unless otherwise mentioned.

Aβ1-42 peptide was dissolved in ice-cold 1,1,1,3,3,3-hexafluoro-2-propanol (HFIP, Sigma-Aldrich) 50 μg or 100 μg aliquots were transferred to siliconized low retention tubes and HFIP was evaporated in the fume hood for 2 hour. After drying under vacuum for 10 minutes in a SpeedVac, aliquots were stored at −20° C. The other peptides were dissolved in 100 mM NH₄CO₃ and lyophilized aliquots were stored at −20° C. Peptide aliquots were freshly dissolved in HPLC grade H₂O at 0.2 mg/ml and together with peptide assembly preparations transferred to 384-well plates and printed immediately. For peptide assembly preparations 100 μg peptide was dissolved in 5 μl freshly opened molecular biology grade DMSO (Sigma-Aldrich, USA) and sonicated for 3 minutes in a water bath.

For oligomeric preparations, solutions were mixed with 195 μl PBS (pH7.2, Ca²⁺/Mg²⁺ free, tissue culture grade) and incubated at 4° C. for 24 hours. For fibrillar preparations, solutions were mixed with 95 μl 110 mM HCl and incubated at 37° C. for 24 hours. Chemically cross-linked species of Aβ1-42 (CAPS) were prepared as described (Mori et al., 1992; Naslund et al., 1994; Saido et al., 1995). Briefly, 100 μg Aβ1-42 was dissolved in 1 ml 50 mM Tris-HCl/150 mM NaCl, mixed with 2 μl of 5 mg/ml horse radish peroxidase and 10 μl freshly made 10 mM H₂O₂. After incubation for 48 hours at 37° C. the reaction was stopped by adding 100 μl 50% NaN₃. Quality of Aβ assemblies was checked routinely by Western blot analysis with specific antibodies or by silver staining.

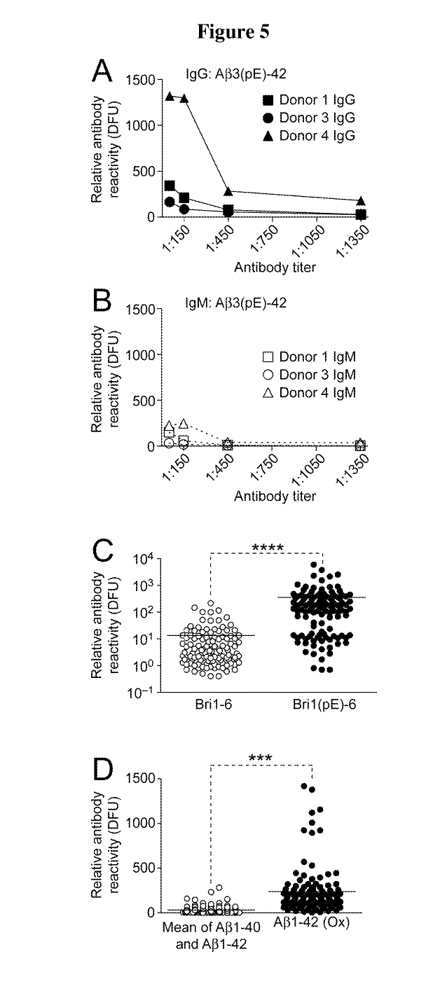

Antigen arrays and data processing. Antigen microarrays (Table 1 and FIG. 5) were printed in batches of up to 200 slides with a robotic microarrayer by contact printing onto reactive epoxide-coated glass slides in an ordered array and the glass slides were probed with diluted human plasma or specific antibodies as described (Robinson et al., 2002). Printed array glass slides were stored in air-tight and sealed bags at 4° C. and used in experiments within two weeks. Arrays were circled by pap pen and glass slides were blocked overnight at 4° C. in 0.45 μm filtered 3% fetal calf serum (FCS) in PBS with 0.5% Tween-20, probed with 1:150 dilutions of human plasma in blocking buffer without Tween for 1 h at 4° C., and bound IgGs were immediately detected by incubation with Alexa Fluor 555- or Alexa Fluor 647-conjugated goat-anti-human IgG (Molecular Probes/Invitrogen).

After incubations slides were washed three times in blocking buffer and finally rinsed in demineralized H₂O, spun in a swing bucket centrifuge for eight minutes at 100 g and scanned at constant PMT with a GenePix 4000B scanner (Molecular Devices, Sunnyvale Calif., USA; GenePix Pro 5.0 software). As positive controls for human IgG reactivity and as control of consistent printing we include at different locations on the array various dilutions of C. albicans extract (Candin®, Allermed Laboratories Inc., San Diego Calif., USA), pneumococcal vaccine (Pneumovax, Merck & Co. Inc., Whitehouse Station N.J., USA), polyvalent, anti-human IgG(H+L) and anti-human IgG/IgM (Jackson Immunoresearch Laboratories Inc., West Grove Pa., USA). Cy3-labeled BSA was printed in each sub-array block (FIG. 5) for better recognition of array orientation and alignment of electronic data extraction template.

Presence of Aβ peptides was verified by probing several glass slides with Aβ-specific antibodies 3D6 (Aβ1-5 specific) and 4G8 (Aβ17-24 specific) and detecting with fluorescently tagged anti-mouse IgG. Slides were scanned at constant PMT to generate false color images on which net median pixel intensities for individual features was measured. For inter-array normalization local background subtracted median values from four antigen features were divided by the median intensity of four to eight features of anti-human-IgG resulting in relative median digital fluorescence units (DFU).

Statistical analyses. All three sample sets were analyzed individually. Most statistical analyses were done in GraphPad Prism 5.0 with p≦0.05 considered significant. Differences between two groups were analyzed by Mann-Whitney U test, for three and more groups one-way ANOVA and Tukey's post hoc test or non-parametric one-way ANOVA Kruskal-Wallis and Dunn's post hoc test were applied. Correlation of MMSE with antibody reactivities was done by Spearman rank method. A trend line with 95% confidence interval (C.I.) was calculated post hoc by linear regression analysis. Multiple comparison analysis between youngest and oldest age group and linear regression analysis of age with z-scored antibody reactivities was done in significance analysis of microarray (SAM) and 0% false discovery rate (FDR) was considered significant. Penalized linear regression models were calculated by the regularization and variable selection method elastic net (hap://cran.r-project.org/web/packages/elasticnet/index.html (Zou & Hastie, 2005). An internal 10-fold cross-validation estimated trade-off parameters and generated a list of best predictors for age. Unsupervised cluster analysis was done in Cluster 3.0 and a node map was generated in Java TreeView.

Antigen-antibody dissociation and ELISA. Dissociation of immune complexes in plasma and detection by indirect ELISA was done at pH 3.5 as described with modifications (Szabo et al., 2008; Li et al., 2007). Freshly prepared oligomer preparation of Aβ1-42 was diluted to 20 μg/ml and 50 μl per well was coated on ELISA plates overnight at 4° C. Plates were blocked with 1.5% BSA (EIA grade, Sigma) and 0.05% Tween-20 in PBS pH 7.0 for 1 hour at 37° C. For dissociation plasma was diluted 1:50 in 0.2 M glycine-HCL in PBS (pH 3.5) with 1.5% BSA and 0.05% Tween-20 and after 20 minute incubation at room temperature 2 M NaOH was added to reconstitute pH 7.0. Untreated plasma was diluted 1:50 in PBS (pH 7.2) with 1.5% BSA and 0.01% Tween-20.

Samples were immediately put on ELISA plates and incubated for 1 hour at room temperature. After five washes with 0.45% NaCl and 0.05% Tween-20, bound IgGs were detected with HRP-tagged anti-human IgG (1:5,000) and after another ten washes the ELISA was developed with 1-Step Turbo TMB substrate (Pierce). The reaction was stopped at 30 minutes with 1M H₂SO₄ and optical density (OD) was measured at 450 nm with an ELISA plate reader. As a control, we titrated 3D6 mAb and measured OD values between 0.1 ng/ml to about 3.0 ng/ml that were comparable to untreated or dissociated plasma.

Immunoblots with human IgGs and Aβ-specific antibody. Preparations of Aβ1-42 peptides were run on 10-20% Trice gels (Invitrogen) and transferred to 0.2 μm nitrocellulose membranes. After 1 hour blocking in TBS/0.01% Tween-20 with 10% milk powder (blotting grade, non-fat dry milk, Biorad), membranes were probed with isolated (Melon™ Gel IgG Spin Purification Kit, Pierce, Rockford Ill., USA) and biotinylated (ProtOn™, Vector Laboratories, Burlingame Calif., USA) human IgGs at 1:200 overnight in TBS/0.01% Tween-20 with 5% milk powder. Bound IgGs were detected by incubating the membrane with 1:20,000 HRP-coupled Avidin D (Vector Laboratories) and blots were developed with ECL chemiluminescence reagents (Amersham Biosciences, Piscataway N.J., USA) and exposed to autoradiography films. In parallel experiments, blots were probed with 0.5 μg/ml Aβ1-5 specific mouse monoclonal antibody (3D6) and detected with HRP-labeled polyclonal anti-mouse IgG (H+L).

Atomic force and electron microscopy. For atomic force microscopy fresh preparations of Aβ1-42 assemblies were diluted to 10 μM and 2 μl were printed by hand onto epoxide-coated microarray glass slides or on Piranha cleaned silicon wafers with molecular smoothness (˜1 Å RMS roughness as determined by AFM 1×1 μm scan) that were modified with epoxy-silane. After 2 minute incubation, samples were washed twice in HPLC grade H₂O and air-dried. AFM topography images were acquired in the light tapping mode regime using a Multimode AFM (Veeco, Santa Barbara Calif., USA). Resonance frequencies of the uncoated silicon tips (MikroMasch, San Jose Calif., USA) were roughly 150 kHz. For electron microscopy 3 μl of oligomer preparation of Bri16-23(A16K/V17K) or peptide freshly solubilized by the same method were placed onto carbon coated 400 mesh Cu/Rh grids (Ted Pella, Inc., Redding, Calif.) and stained with 1% uranyl acetate in distilled water (Polysciences, Inc., Warrington, Pa.). Stained grids were examined in a Philips CM-12 transmission electron microscope and photographed with a Gatan (4 k×4 k) digital camera at the Image Core Facility of the Skirball Institute of Biomedical Medicine, NYU School of Medicine.

The following examples are put forth so as to provide those of ordinary skill in the art with a complete disclosure and description of how to make and use the subject invention, and are not intended to limit the scope of what is regarded as the invention. Efforts have been made to ensure numerical accuracy.

### EXAMPLES

### Example 1

### Plasma Aβ Antibodies Predominantly Recognize High Molecular Weight Assemblies in Oligomeric Preparations of Aβ1-42

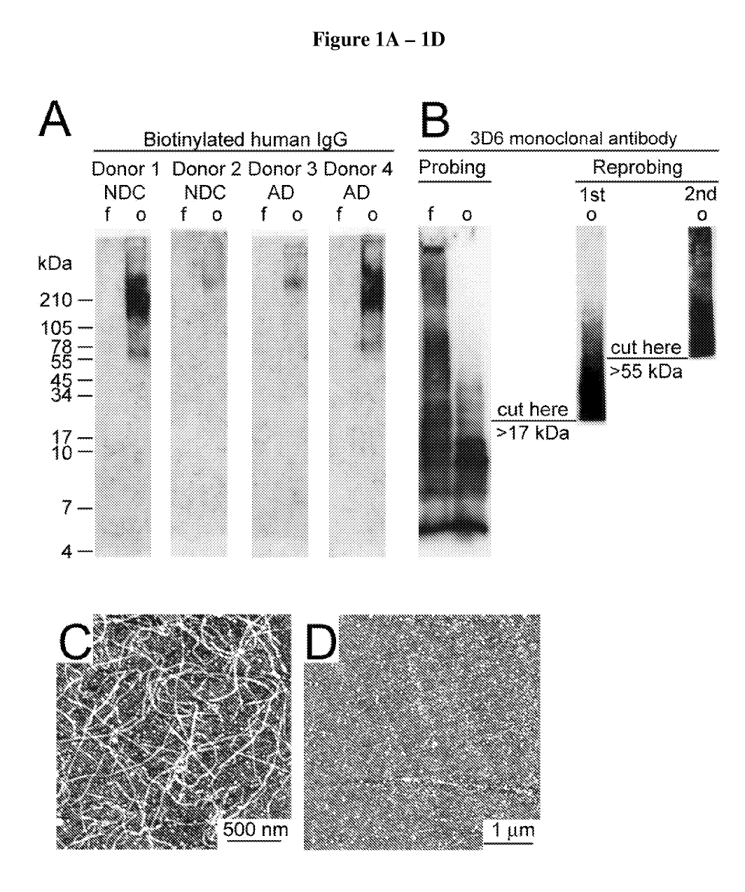

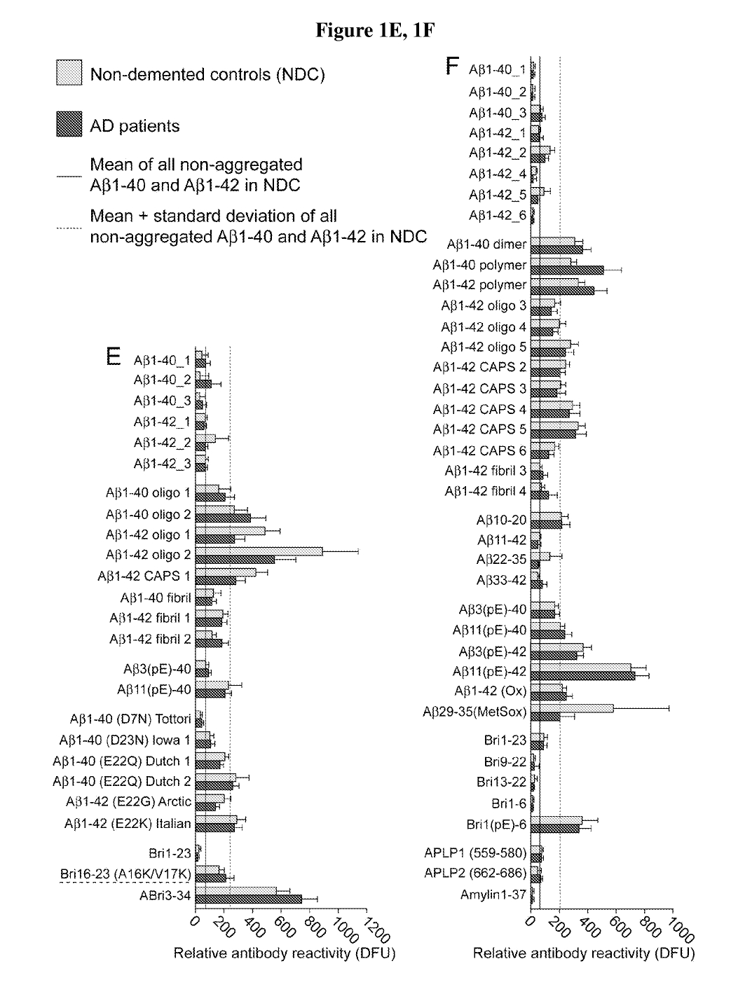

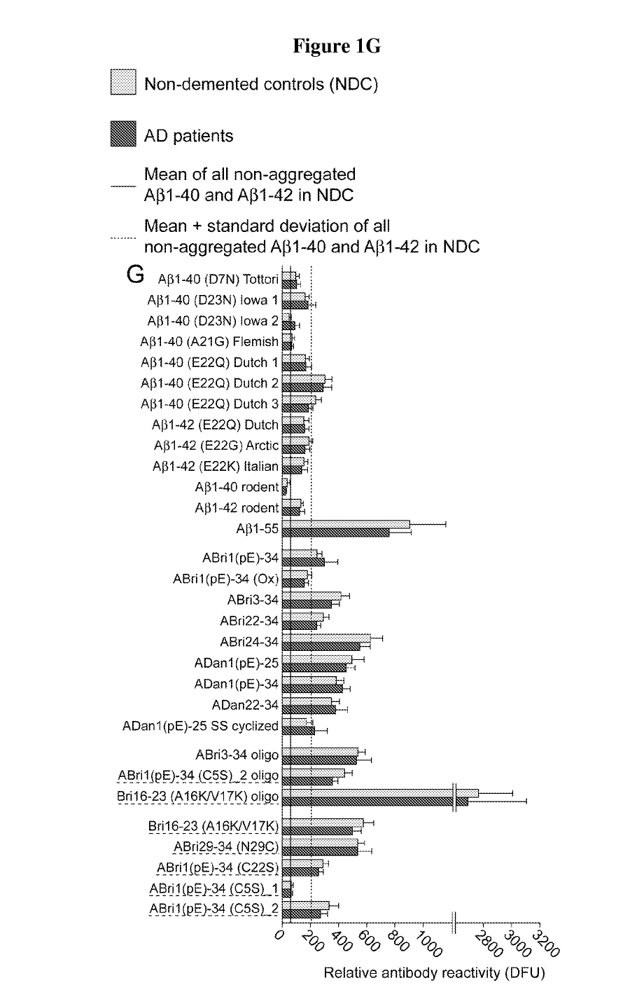

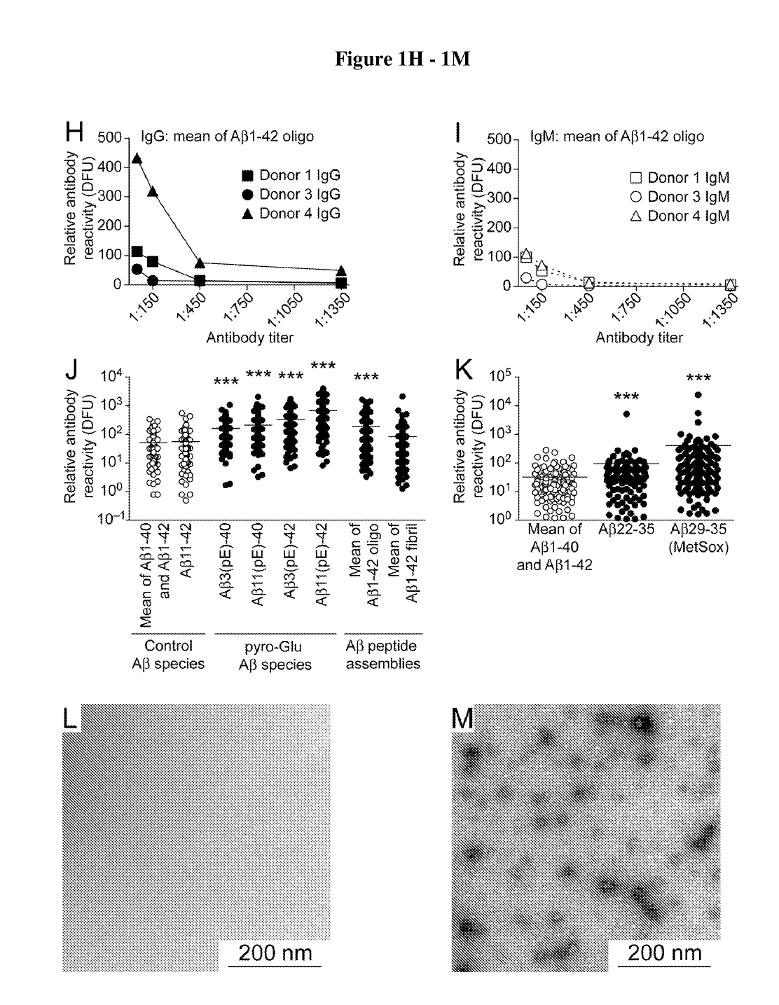

To characterize the specificity of human plasma Aβ antibodies we isolated IgGs, and analyzed their reactivity to assemblies of Aβ1-42 peptides on Western blots. Some IgG samples reacted distinctively with a nonfibrillar Aβ assembly between 55-78 kDa, reminiscent of the previously described 56 kDa Aβ*56 (Lesne et al., 2006) but the main reactivity in all samples was against entities larger than 210 kDa (FIG. 1A). The lack of binding to fibrillar Aβ preparations (FIG. 1A) indicates that most of the IgG antibodies are specific to smaller, potentially oligomeric conformations. As a control, the Aβ1-5 specific monoclonal antibody 3D6 preferentially bound to highly abundant monomers, dimers and trimers in the oligomeric as well as in the fibrillar preparation and also to higher molecular weight assemblies in the fibrillar preparation on the same blot (FIG. 1B).

We detected the high molecular weight assemblies in the oligomeric Aβ preparation only after repeated and isolated reprobing of the respective area of the membrane indicating that these larger assemblies are likely present at low levels (FIG. 1B). Atomic force microscopy confirmed the presence of predominantly fibrillar structures in fibrillar preparations (FIG. 1C) or nonfibrillar Aβ species in oligomeric preparations of Aβ1-42 (FIG. 1D). Together these data demonstrate the existence of highly specific IgG antibodies recognizing conformational epitopes in high molecular weight Aβ1-42 assemblies present in human plasma.

### Example 2

### Plasma Aβ Antibodies are Abundant and Directed Preferentially Against Oligomers and Post-Translationally Modified Aβ

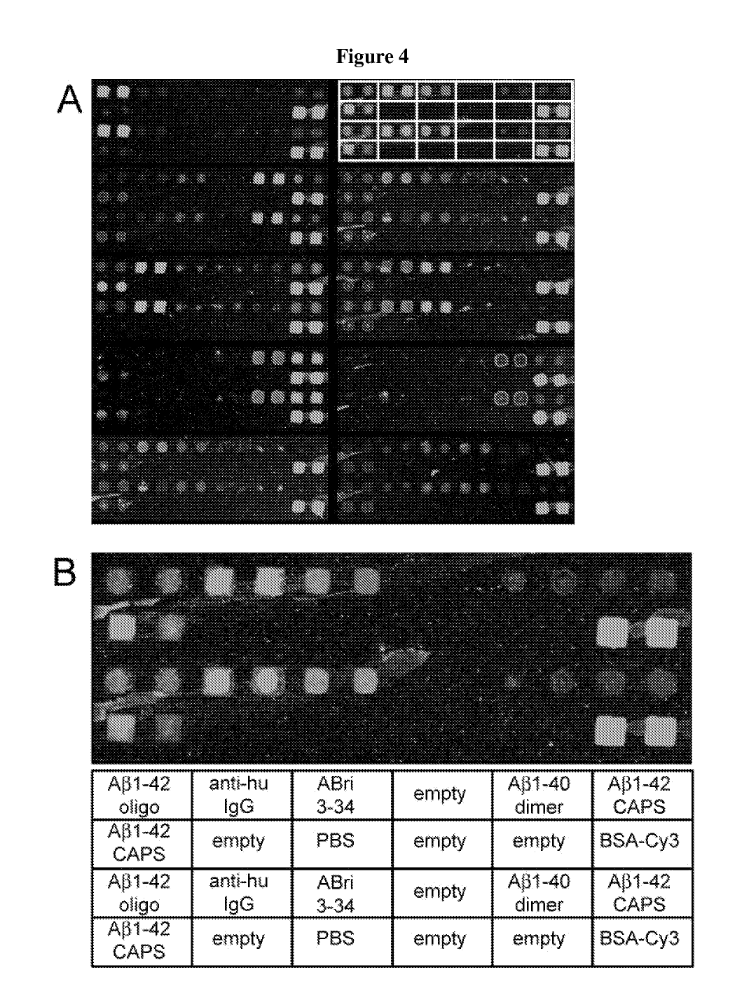

To characterize and quantify the repertoire of natural antibodies against Aβ in human plasma on a large scale, we developed peptide microarrays containing various synthetic Aβ peptide variants, either non-aggregated or in different assembly states, as well as non-A13 amyloidogenic peptides and controls (Table 1 and FIG. 4). To mimic some effects of oxidative stress in vitro we generated di-tyrosine cross-linked Aβ peptide species (CAPS) (Mori et al., 1992) and confirmed oligomerization by Western blot analysis (data not shown). In a first experiment we measured relative Aβ antibody reactivities of the IgG class against 25 different peptide preparations (Table 1) in plasma from 75 AD patients at various stages of disease and 36 healthy non-demented controls (NDC) (Table 2). Strikingly, antibodies recognizing higher order Aβ assemblies, including oligomer and fibrillar preparations, were more prevalent and up to 12-fold higher than the average reactivity against presumably non-aggregated preparations of Aβ1-40 and Aβ1-42 (FIG. 1E). No significant differences were observed between plasma from this mixed group of AD patients and NDC. Besides antibodies against conformational Aβ species we also discovered antibody reactivities against pyroglutamate Aβ variants, and curiously, against the peptide residue ABri3-34 (FIG. 1E).

To replicate these findings in an independent sample set and to extend the panel of peptides, we screened plasma samples from an additional sample set of 55 AD patients and 62 NDC subjects (Table 2) for antibodies against 74 different peptide preparations and controls (Table 1 and FIG. 1F, G). We confirmed that IgG reactivities against Aβ assemblies were generally higher than reactivities against non-aggregated Aβ (FIG. 1F).

Although IgG titers to oligomeric preparations of Aβ1-42 (FIG. 1H) and other peptides (FIG. 5A, and data not shown) are relatively low, they were 2- to 4-fold higher than IgM reactivities within the same plasma sample (FIG. 1I) and for certain peptides up to 86-fold higher (FIG. 5A, B). These antibody titers are also consistent with peptide array-based measurements of IgGs reactive to Aβ and myelin proteins in patients with multiple sclerosis (Quintana et al., 2008). and support the concept of a circulating pool of self-reactive IgG antibodies in the general population (Tiller et al., 2007; Lutz et al., 2009).

Interestingly, most plasma samples had relatively high IgG antibody reactivity against posttranslationally modified peptides. For example, IgGs against pyroglutamate variants Aβ3(pE)-42 or Aβ11(pE)-42 were up to 12-fold higher than those against Aβ11-42 or the average reactivities against Aβ1-40 and Aβ1-42 (FIG. 1J). Similarly, the pyroglutamate form of Bril(pE)-6 elicited a 26-fold higher reactivity than Bril-6 (FIG. 5C).

Oxidative modification of Aβ peptide as present in Aβ29-M35(MetSox) (FIG. 1K) or Aβ1-42 (Ox) (FIG. 5D) led to a prominent increase in IgG binding compared with the median IgG reactivity to non-aggregated Aβ1-40 and Aβ1-42. In contrast, antibody reactivities against control peptides APLP1 (559-580), APLP2 (662-686), or human Amylin1-37 were similar to reactivities against non-aggregated Aβ1-40 or Aβ1-42 peptides (FIG. 1F).

### Example 3

### General Population Carries Relatively High Antibody Reactivity Against Amyloidogenic Peptides Unique to Autosomal Dominant Dementias

Of particular interest in the first experiment was the increased antibody reactivity against mutant Aβ peptides that are found exclusively in different familial forms of AD or against the peptide residue ABri3-34 (FIG. 1E), a putative fragment of an amyloidogenic peptide uniquely present in patients with familial British dementia. Consistent with the first experiment, relative antibody reactivities to mutant forms of Aβ1-40 or Aβ1-42 were several-fold increased compared to median reactivity for non-aggregated preparations of Aβ1-40 or Aβ1-42 (FIG. 1G), and 15.2-fold higher for Aβ1-55 (FIG. 1G).

We observed relatively high antibody reactivities against full-length ABri and measured similar IgG titers for ADan (FIG. 1G), both peptides being mutant amyloidogenic non-Aβ peptides present exclusively either in British (Vidal et al., 1999) or Danish dementia (Vidal et al., 2000). Oligomer preparations and posttranslationally modified residues or artificial variants of same peptides typically elicited even higher reactivities, e.g. relative reactivity against an oligomeric preparation of artificial mutant Bri16-23(A16K/V17K) was the strongest observed in our study (FIG. 1G). Electron microscopy confirmed in this preparation the presence of structures reminiscent of previously described globular or annular assemblies of Aβ and other amyloidogenic peptides (Lashuel et al., 2002; Quist et al., 2005) but no assemblies were detected in freshly dissolved peptide (FIG. 1 L, M). Together, these data from two independent sample sets demonstrate for the first time the abundance of a diverse IgG repertoire in human plasma recognizing higher order assemblies of Aβ peptides, pyroglutamate Aβ, oxidized Aβ, and mutant Bri peptide residues. The presence of antibodies in individuals who are not carriers of a mutation for familial dementia supports the existence of cross-reactive conformation-specific antibodies.

### Example 4

### Reduced Antibody Reactivities Against Oligomeric Aβ1-42 Assemblies in Advanced AD and in Normal Aging

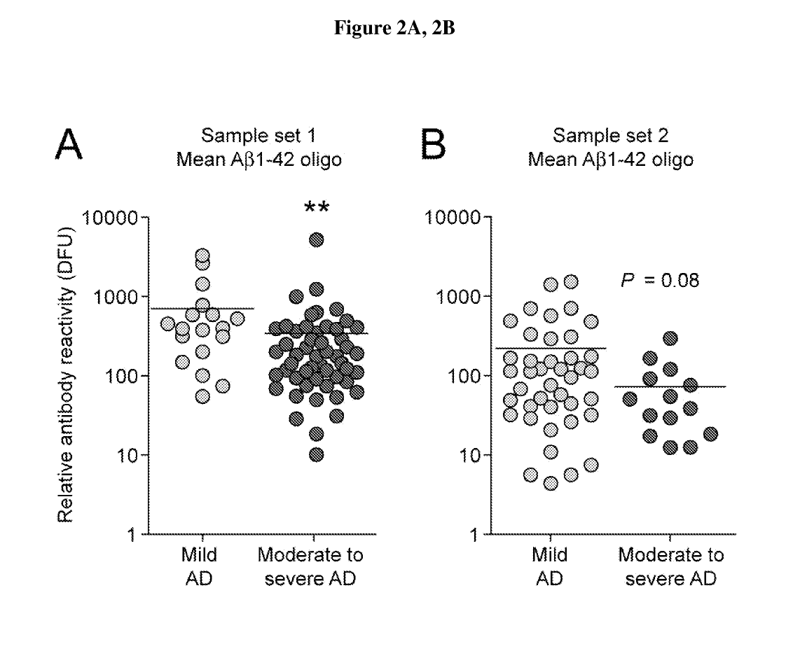

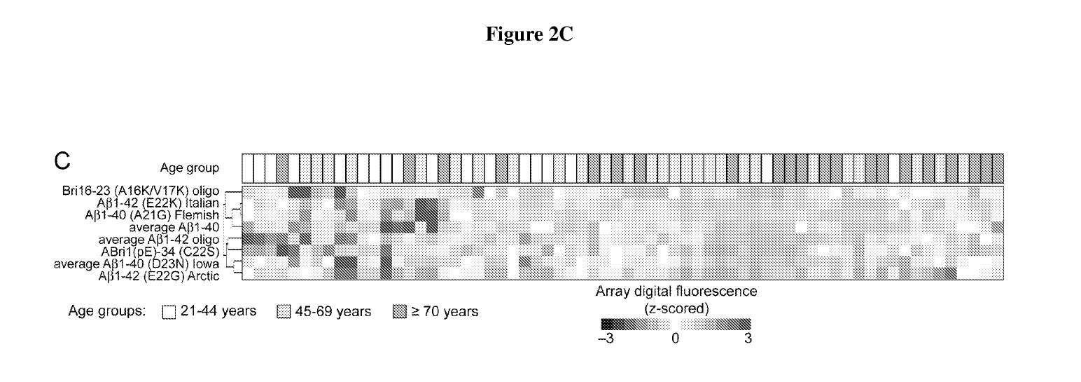

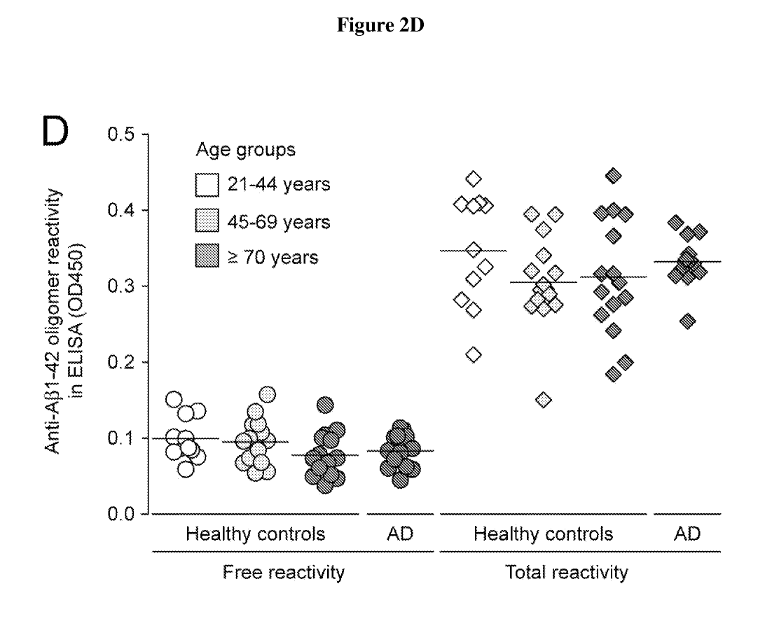

Although no overall differences in Aβ or other peptide antibody reactivities were observed between a diverse group of AD patients and NDC, patients in sample set 1 with moderate to severe AD (mini-mental state exam score, MMSE scores ≦19) had significantly lower levels of antibody reactivities against oligomeric preparations of Aβ1-42 than patients with mild disease (MMSE scores ≧20) (FIG. 2A). A similar group difference was found in sample set 2, although it did not reach significance (FIG. 2B), possibly due to the low number of patients with severe disease (Table 2). No group difference was detected for antibodies recognizing non-aggregated Aβ, control antigens ‘pneumococcal vaccine polyvalent’ and C. albicans extract (data not shown), arguing against a general age-related decline in antibody titers in our sample sets. Also, ApoE4 carriers did not differ from non-carriers in their immunoreactivity with all tested antigens (data not shown).

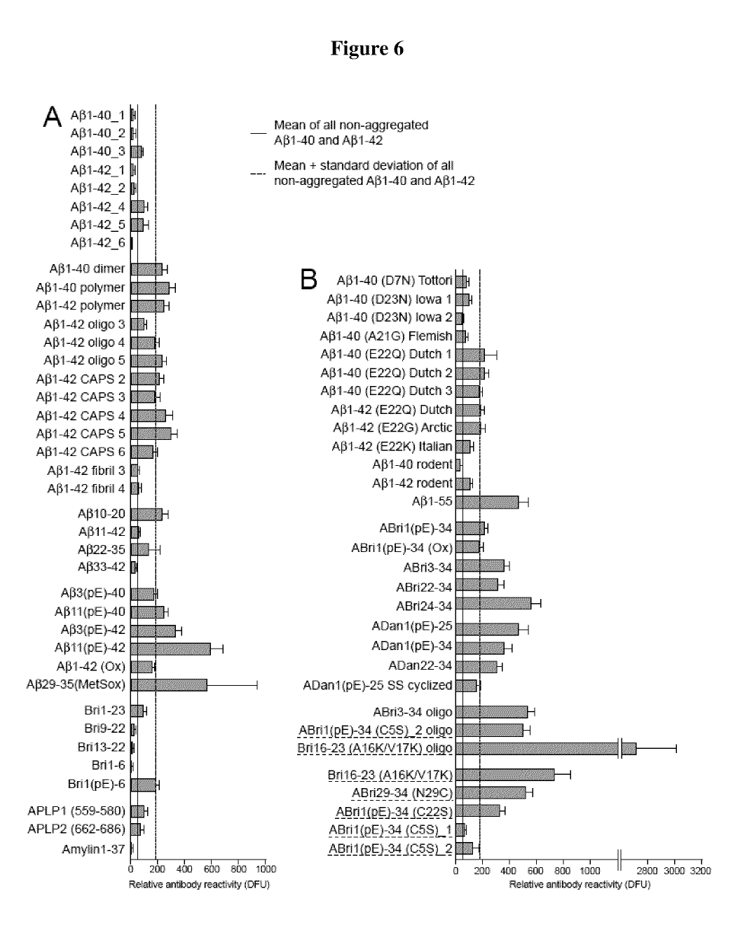

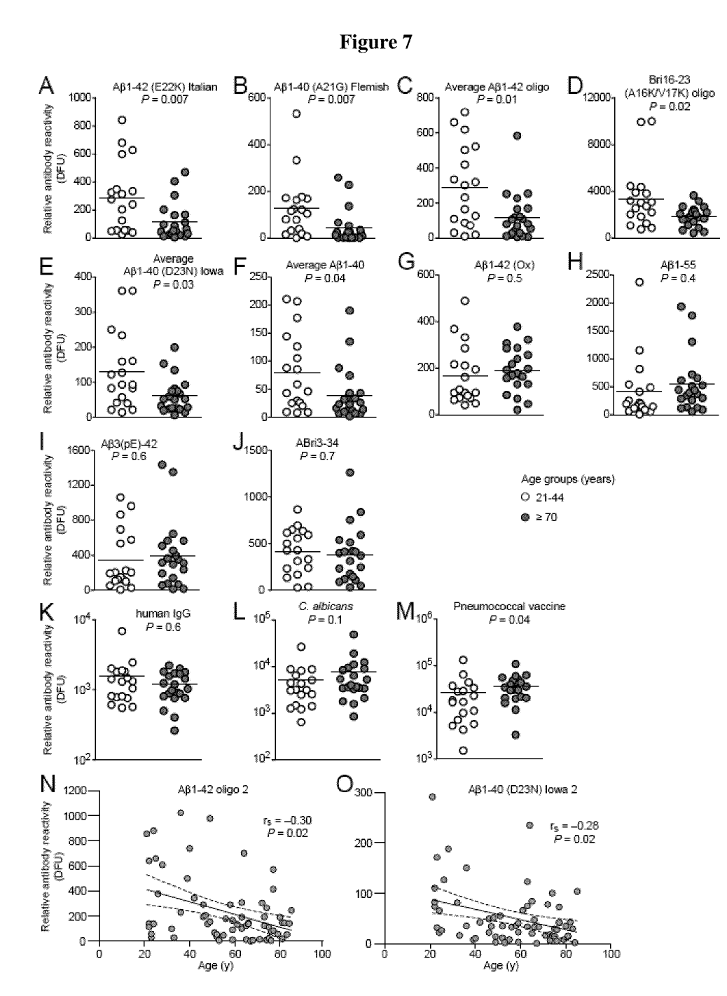

To determine antibody reactivities at different ages we screened plasma samples from a third set of samples collected from 66 healthy, non-demented females ages 21 to 85 years old (Table 2 and FIG. 6) and found six antibody reactivities that were on average 45-65% higher in plasma from the youngest (21-44 years) compared with the oldest (≧70 years) age groups (FIG. 7A-F). Total IgG levels and other reactivities did not change (FIG. 7G-L and data not shown), and only reactivity to pneumococcal vaccine increased with age (FIG. 7M) again arguing against a general loss in overall immunity in the elderly studied here.

Using linear regression methods we found eight antibody reactivities that were strongest associated with age as illustrated in a node map generated by an unsupervised cluster algorithm to arrange all samples based on similarity of antibody reactivities (FIG. 2C). Antibody reactivities against some of these peptides showed significant inverse correlations with age (FIG. 7N, O).

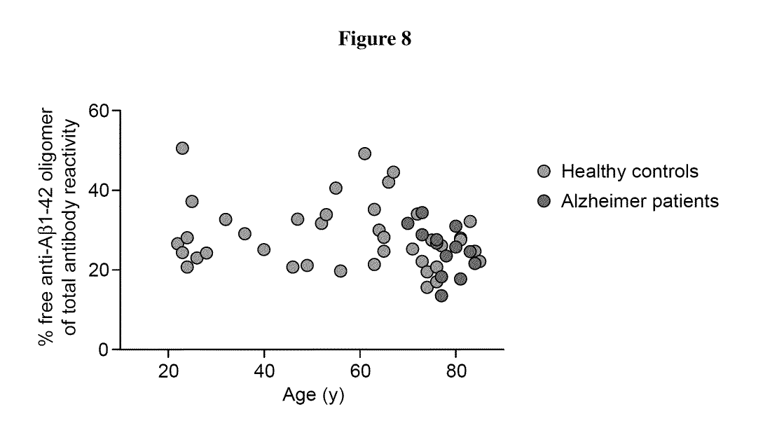

A comparison of reactivities of free Aβ antibodies with pH 3.5 immune complex-dissociated Aβ antibodies (Li et al., 2007) by ELISA showed that low pH treatment increased antibody reactivities against oligomeric Aβ in all tested plasma samples 2 to 7.4-fold (FIG. 2D, mean 3.9±1.1 standard deviation) compared with untreated samples. This is similar to reports in human serum and IvIg preparations (Szabo et al., 2008; Gustaw et al., 2008). The percentage of free antibodies out of the total pool recognizing Aβ1-42 oligomers ranged from 13% to 50% but was not associated with age or AD (mean 27.7%±7.8% standard deviation; FIG. 8). Together, these data indicate that relative plasma antibody reactivities against several amyloidogenic Aβ mutant and non-Aβ peptides and in particular to oligomeric Aβ1-42 are reduced with age.

### Example 5

### Plasma IgG Reactivity is Similar in CSF and Protects Primary Neurons from Aβ Toxicity

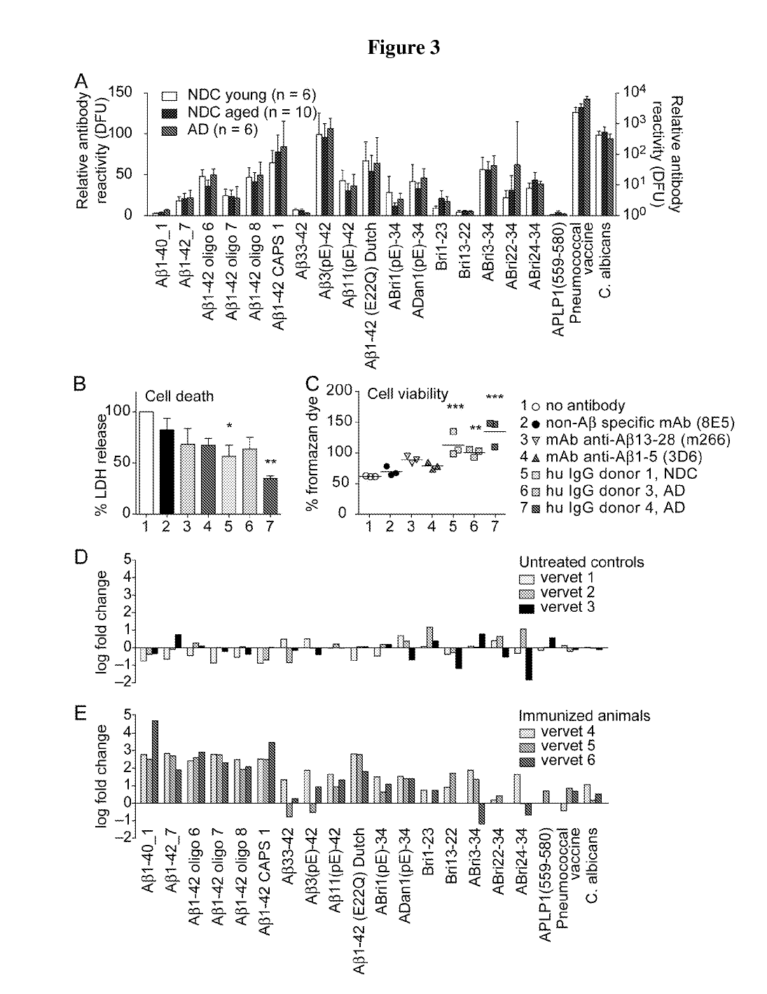

To investigate whether any of the antibodies detected in the periphery may have access to the CNS we measured antibody titers in CSF samples from healthy normal controls (n=16, aged 24-83 years) and patients with mild AD (n=6, aged 70-84 years) using peptide arrays. Average reactivities in CSF were about 30 to 230 times lower in CSF than in plasma but a similar repertoire of IgG antibodies was seen. Similar to plasma, highest titers were observed for oligomeric preparations of Aβ1-42, pyroglutamate modified Aβ peptides, and mutant variants of Aβ, ABri and ADan (FIG. 3A). There was no significant difference in reactivities in the analyzed sample groups and IgM was not detectable (data not shown).

To assess the potential physiological relevance of natural Aβ antibodies we tested the protective potential of IgGs against Aβ toxicity. IgGs purified from three plasma samples and acidified to release bound peptides (see above) were preincubated with oligomeric preparations of Aβ1-42 and this mix was added to primary hippocampal neurons from E16 mice. All three IgG preparations significantly reduced cell death (FIG. 3B) and increased cell viability (FIG. 3C) to an equal or greater extent than monoclonal antibodies to Aβ, consistent with similar experiments done with IvIg (Szabo et al., 2008).

### Example 6

### Immunization with Aβ Peptides Amplifies Immune Response to Aβ, ABri, and ADan Peptides in Non-Human Primates

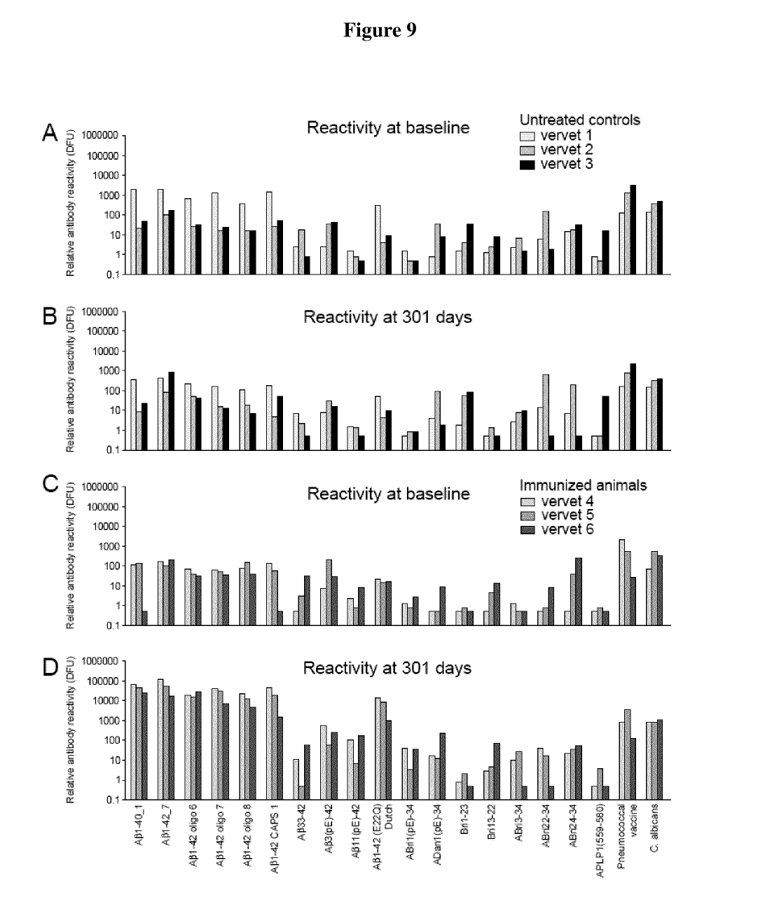

To further study the in vivo relevance of natural antibodies to amyloidogenic peptides, we measured immunoreactivity in plasma of a sub-cohort of vervet monkeys from a previously published study (Lernere et al., 2004). Aged animals can develop features of AD including cerebral amyloid plaques that stain positive with anti-human Aβ antibodies (Lernere et al., 2004) indicating high homology between human and vervet Aβ similar to other non-human primates (Podlisny et al., 1991). Immunization with a cocktail of human Aβ1-40 and Aβ1-42 almost entirely cleared cerebral Aβ plaques (Lernere et al., 2004). While standard ELISA methods detected miniscule levels of Aβ1-40 antibodies in non-immunized vervets (Lernere et al., 2004), using our peptide arrays, we were able to measure reactivities to non-aggregated and oligomeric Aβ preparations as well as to Aβ3(pE)-42 species comparable to what we detected in human plasma (FIG. 9A-C).

At the end of the study, at 301 days, control animals had unchanged or decreased titers to most of the tested peptides (FIG. 3D, FIG. 9B), while immunized vervets developed a 370-fold median increase (range of 80 to 50000-fold) in IgG antibodies to full length Aβ preparations (FIG. 3E, FIG. 9D). In support of earlier findings that immunization with Aβ was mainly targeting the peptide's N-terminus (Lernere et al., 2004) immunized vervets failed to develop a comparable immune response to Aβ33-42 (0.2 to 22-fold).

Most importantly, and in likely support of their physiological relevance, immunized vervets significantly increased antibody levels to pyroglutamate modified Aβ (e.g. Aβ11(pE)-42), mutant Aβ (e.g. Aβ1-42(E22Q) Dutch), and the foreign peptides ABri and ADan (FIG. 3E, FIG. 9D). These data show that immunization with full-length Aβ not only increases immunity to select Aβ peptide species but also triggers the expansion of cross-reactive antibodies against other known toxic amyloidogenic peptides.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

### REFERENCES

Bard et al. (2003). “Epitope and isotype specificities of antibodies to beta-amyloid peptide for protection against Alzheimer's disease-like neuropathology.” Proc. Natl. Acad. Sci. USA 100, pp. 2023-2028
Bateman et al. (2006). “Human amyloid-beta synthesis and clearance rates as measured in cerebrospinal fluid in vivo”. Nat. Med. 12, pp. 856-861
Britschgi et al. (2009). “Neuroprotective natural antibodies to assemblies of amyloidogenic peptides decrease with normal aging and advancing Alzheimer's disease.” Proc. Natl. Acad. Sci. USA 106, pp. 12145-12150
Deane and Zlokovic (2007). “Role of the Blood-Brain Barrier in the Pathogenesis of Alzheimer's Disease.” Curr. Alzheimer Res. 4, pp. 191-197
DeMattos et al. (2001). “Peripheral anti-Aβ antibody alters CNS and plasma Aβ clearance and decreases brain Aβ burden in a mouse model of Alzheimer's disease.” Proc. Natl. Acad. Sci. USA 98, pp. 8850-8855
Dodel et al. (2002). “Human antibodies against amyloid β peptide: A potential treatment for Alzheimer's disease”. Ann. Neurol. 52, pp. 253-256
Du et al. (2001). “Reduced levels of amyloid β-peptide antibody in Alzheimer disease”. Neurology 57, pp. 801-805
Dubois et al. (2007). “Research criteria for the diagnosis of Alzheimer's disease: revising the NINCDS-ADRDA criteria”. Lancet Neurol. 6, pp. 734-746
Folstein et al. (1975) “Mini-mental state”—A practical method for grading the cognitive state of patients for the clinician. J. Psychiatr. Res. 12, pp. 189-198
Gaskin et al. (1993). “Human antibodies reactive with beta-amyloid protein in Alzheimer's disease”. J. Exp. Med. 177, pp. 1181-1186
Gilman et al. (2005). “Clinical effects of Aβ immunization (AN1792) in patients with AD in an interrupted trial”. Neurology 64, pp. 1553-1562
Gruden et al. (2007). “Differential neuroimmune markers to the onset of Alzheimer's disease neurodegeneration and dementia: autoantibodies to Abeta ((25-35)) oligomers, S100b and neurotransmitters”. J. Neuroimmunol. 186, pp. 181-192
Gustaw et al. (2008). “Antigen-antibody dissociation in Alzheimer disease: a novel approach to diagnosis.” J. Neurochem. 106, pp. 1 1350-1356
Haass & Selkoe (2007). “Soluble protein oligomers in neurodegeneration: lessons from the Alzheimer's amyloid β-peptide.” Nat. Rev. Mol. Cell. Biol. 8, pp. 101-112
Henkel et al. (2007). “Immune complexes of auto-antibodies against A beta 1-42 peptides patrol cerebrospinal fluid of non-Alzheimer's patients.” Mol. Psychiatry. 12, pp. 601-610
Hyman et al. (2001). “Autoantibodies to amyloid-beta in Alzheimer's disease.” Ann. Neurol. 49, pp. 808-8010
Jianping et al. (2006). “Low avidity and level of serum anti-Abeta antibodies in Alzheimer disease.” Alzheimer Dis. Assoc. Disord. 20, pp. 127-132
Klyubin et al. (2005). “Amyloid β protein immunotherapy neutralizes Aβ oligomers that disrupt synaptic plasticity in vivo.” Nat. Med. 11, pp. 556-561
Lashuel et al. (2002). Amyloid pores from pathogenic mutations. Nature 418, p 291
Levites et al. (2006). “Anti-Aβ42- and anti-Aβ40-specific mAbs attenuate amyloid deposition in an Alzheimer disease mouse model.” J. Clin. Invest. 116, pp. 193-201
Lemere C A et al. (2004). “Alzheimer's Disease Aβ Vaccine Reduces Central Nervous System Aβ Levels in a Non-Human Primate, the Caribbean Vervet.” Am. J. Pathol. 165, pp. 283-297
Lesne et al. (2006). “A specific amyloid-b protein assembly in the brain impairs memory”, Nature 440, pp. 352-357
Li Q et al. (2007). “Improvement of a low pH antigen-antibody dissociation procedure for ELISA measurement of circulating anti-Aβ antibodies. BMC Neurosci. 8, p. 22
Lutz et al. (2009). “Naturally occurring auto-antibodies in homeostasis and disease”. Trends Immunol 30, pp. 43-51
Mamikonyan et al. (2007). “Anti-Aβ₁₋₁₁ Antibody Binds to Different β-Amyloid Species, Inhibits Fibril Formation, and Disaggregates Preformed Fibrils but Not the Most Toxic Oligomers”. J. Biol. Chem. 282, pp. 22376-22386
Moir et al. (2005). “Autoantibodies to redox-modified oligomeric Abeta are attenuated in the plasma of Alzheimer's disease patients.” J. Biol. Chem. 280, pp. 17458-17463
Mori et al. (1992). “Mass Spectrometry of Purified Amyloid β Protein in Alzheimer's Disease.” J. Biol. Chem. 267, pp. 17082-17086
Mruthinti et al. (2004). “Autoimmunity in Alzheimer's disease: increased levels of circulating IgGs binding Abeta and RAGE peptides.” Neurobiol. Aging 25, pp. 1023-1032
Naslund et al. (1994). “Relative abundance of Alzheimer A beta amyloid peptide variants in Alzheimer disease and normal aging”. Proc. Natl. Acad. Sci. USA 91, pp. 8378-8382
Nath et al. (2003). “Autoantibodies to amyloid beta-peptide (Abeta) are increased in Alzheimer's disease patients and Abeta antibodies can enhance Abeta neurotoxicity: implications for disease pathogenesis and vaccine development.” Neuromol. Med. 3, pp. 29-39
Nitsch & Hock (2008). “Targeting β-Amyloid Pathology in Alzheimer's Disease with Aβ Immunotherapy.” Neurotherapeutics 5, pp. 415-420
Orgogozo et al. (2003). “Subacute meningoencephalitis in a subset of patients with AD after Aβ42 immunization”. Neurology 61, pp. 46-54
Pfeifer et al. (2002). “Cerebral Hemorrhage After Passive Anti-Aβ Immunotherapy”. Science 298, p. 1379
Piccini et al. (2005). “β-Amyloid Is Different in Normal Aging and in Alzheimer Disease”. J. Biol. Chem. 280, pp. 34186-34192
Podlisny et al. (1991). “Homology of the amyloid beta protein precursor in monkey and human supports a primate model for beta amyloidosis in Alzheimer's disease.” Am. J. Pathol. 138, pp. 1423-1435
Quintana et al. (2008). “Antigen microarrays identify unique serum autoantibody signatures in clinical and pathologic subtypes of multiple sclerosis”.
Proc. Natl. Acad. Sci. USA 105, pp. 18889-18894.
Quist et al. (2005). “Amyloid ion channels: A common structural link for protein-misfolding disease”. Proc. Natl. Acad. Sci. USA 102, pp. 10427-10432
Robinson et al. (2002). “Autoantigen microarrays for multiplex characterization of autoantibody responses”. Nat. Med. 8, pp. 295-301
Saido et al. (1995). “Dominant and differential deposition of distinct β-amyloid peptide species, Aβ_{N3(pE)}, in senile plaques.” Neuron 14, pp. 457-466
Schenk et al. (1999). “Immunization with amyloid-β attenuates Alzheimer-disease-like pathology in the PDAPP mouse.” Nature 400, pp. 173-177
Stine et al. (2003). “In Vitro Characterization of Conditions for Amyloid-β Peptide Oligomerization and Fibrillogenesis.” J. Biol. Chem. 278, pp. 11612-11622
Szabo et al. (2008). “Natural human antibodies to amyloid beta peptide.” Autoimmun. Rev. 7, pp. 415-420
Thal et al. (2006). “The Development of Amyloid-β Protein Deposits in the Aged Brain”. Sci. Aging Knowl. Environ. 6, p. re1
Tiller et al. (2007). “Autoreactivity in Human IgG+ Memory B Cells”. Immunity 26, pp. 205-213
Vidal et al. (1999). “A stop-codon mutation in the BRI gene associated with familial British dementia”. Nature 399, pp. 776-781
Vidal et al. (2000). “A decamer duplication in the 3′ region of the BRI gene originates an amyloid peptide that is associated with dementia in a Danish kindred”.
Proc. Natl. Acad. Sci. USA 97, pp. 4920-4925
Weksler et al. (2002). “Patients with Alzheimer disease have lower levels of serum anti-amyloid peptide antibodies than healthy elderly individuals.”
Exp. Gerontol. 37, pp. 943-948
Zhou et al. (2005). “Novel Aβ peptide immunogens modulate plaque pathology and inflammation in a murine model of Alzheimer's disease”. J. Neuroinflammation 2, p. 28
Zou & Hastie (2005). “Regularization and variable selection via the elastic net.” J. Royal Stat. Society 67, pp. 301-320.

What is claimed is:

1. A method of treating a subject with a neurodegenerative pathological condition, organ limited or systemic amyloidosis, said method comprising administering an effective dose of a peptide selected from SEQ ID NOS 3-33 in order to induce an immune response.

2. The method according to claim 1, wherein the peptide can be in monomeric or multimeric assembly structure.

3. The method according to claim 1, wherein said neurodegenerative pathological condition is Alzheimer's disease, Parkinson's disease, Lewy body dementia, Huntington's disease, tauopathy, synucleinopathy, prion disease, or neurodegenerative diseases with TDP-43 aggregates.

4. The method according to claim 1, wherein said organ limited amyloidosis is cardiac amyloidosis.

5. A method of treating a subject with a neurodegenerative pathological condition, organ limited or systemic amyloidosis, said method comprising administering an effective dose of an antibody specific for a peptide selected from SEQ ID NOS 3-33.

6. The method according to claim 5, wherein the peptide can be in monomeric or multimeric assembly structure.

7. The method according to claim 5, wherein said neurodegenerative pathological condition is Alzheimer's disease, Parkinson's disease, Lewy body dementia, Huntington's disease, tauopathy, synucleinopathy, prion disease, or neurodegenerative diseases with TDP-43 aggregates.

8. The method according to claim 5, wherein said organ limited amyloidosis is cardiac amyloidosis.

9. A method of assessing the risk of a subject to develop an amyloidogenic peptide or protein-related disease, said method comprising quantifying the expression of an antibody specific for a peptide selected from SEQ ID NOS 1-33.

10. The method according to claim 9, wherein the peptide can be in monomeric or multimeric assembly structure.

11. The method according to claim 9, wherein said amyloidogenic peptide-related disease is Alzheimer's disease, Parkinson's disease, Lewy body dementia, Huntington's disease, tauopathy, synucleinopathy, prion disease, cardiac amyloidosis, or neurodegenerative diseases with TDP-43 aggregates.

12. A method of diagnosing a subject with an amyloidogenic peptide or protein-related disease, said method comprising detecting an antibody specific for a peptide selected from SEQ ID NOS 1-33.

13. The method according to claim 12, wherein the peptide can be in monomeric or multimeric assembly structure.

14. The method according to claim 12, wherein said amyloidogenic peptide or protein-related disease is Alzheimer's disease, Parkinson's disease, Lewy body dementia, Huntington's disease, tauopathy, synucleinopathy, prion disease, cardiac amyloidosis, or neurodegenerative diseases with TDP-43 aggregates.

15. An isolated antibody that specifically binds to an antigen selected from SEQ ID NOS 3-33.

16. The isolated antibody of claim 15, wherein said isolated antibody is a monoclonal antibody.

17. The isolated antibody of claim 15, wherein said isolated antibody is a polyclonal antibody.

18. The isolated antibody of claim 15, wherein said isolated antibody is a humanized antibody.

19. The isolated antibody of claim 15, wherein said humanized antibody comprises a humanized heavy chain and a humanized light chain.

20. The isolated antibody of claim 15, wherein said isolated antibody is part of an immunotoxin.

21. A pharmaceutical composition comprising an isolated antibody according to claim 15, together with a pharmaceutically acceptable vehicle, excipient and/or diluent.


SEQUENCE LISTING (33 sequences)

SEQ ID NO: 1 - protein, 40 aa, Artificial Sequence
  Synthetic peptide
DAEFRHDSGYEVHHQKLVFFAEDVGSNKGAIIGLMVGGVV

SEQ ID NO: 2 - protein, 42 aa, Artificial Sequence [sequence omitted]

SEQ ID NO: 3 - protein, 10 aa, Artificial Sequence
  Synthetic peptide
YEVHHQKLVF

SEQ ID NO: 4 - protein, 42 aa, Artificial Sequence [sequence omitted]

SEQ ID NO: 5 - protein, 7 aa, Artificial Sequence
  Synthetic peptide
GAIIGLM

SEQ ID NO: 6 - protein, 38 aa, Artificial Sequence
  Synthetic peptide
EFRHDSGYEVHHQKLVFFAEDVGSNKGAIIGLMVGGVV

SEQ ID NO: 7 - protein, 30 aa, Artificial Sequence
  Synthetic peptide
EVHHQKLVFFAEDVGSNKGAIIGLMVGGVV

SEQ ID NO: 8 - protein, 40 aa, Artificial Sequence
  Synthetic peptide
EFRHDSGYEVHHQKLVFFAEDVGSNKGAIIGLMVGGVVIA

SEQ ID NO: 9 - protein, 32 aa, Artificial Sequence
  Synthetic peptide
EVHHQKLVFFAEDVGSNKGAIIGLMVGGVVIA

SEQ ID NO: 10 - protein, 40 aa, Artificial Sequence
  Synthetic peptide
DAEFRHDSGYEVHHQKLVFFAQDVGSNKGAIIGLMVGGVV

SEQ ID NO: 11 - protein, 42 aa, Artificial Sequence
  Synthetic peptide
DAEFRHDSGYEVHHQKLVFFAQDVGSNKGAIIGLMVGGVVIA

SEQ ID NO: 12 - protein, 42 aa, Artificial Sequence
  Synthetic peptide
DAEFRHDSGYEVHHQKLVFFAGDVGSNKGAIIGLMVGGVVIA

SEQ ID NO: 13 - protein, 42 aa, Artificial Sequence
  Synthetic peptide
DAEFRHDSGYEVHHQKLVFFAKDVGSNKGAIIGLMVGGVVIA

SEQ ID NO: 14 - protein, 6 aa, Artificial Sequence
  Synthetic peptide
EASNCF

SEQ ID NO: 15 - protein, 8 aa, Artificial Sequence
  Synthetic peptide
KKETLICS

SEQ ID NO: 16 - protein, 34 aa, Artificial Sequence
  Synthetic peptide
EASNCFAIRHFENKFAVETLICSRTVKKNIIEEN

SEQ ID NO: 17 - protein, 31 aa, Artificial Sequence
  Synthetic peptide
NCFAIRHFENKFAVETLICSRTVKKNIIEEN

SEQ ID NO: 18 - protein, 13 aa, Artificial Sequence
  Synthetic peptide
CSRTVKKNIIEEN

SEQ ID NO: 19 - protein, 11 aa, Artificial Sequence
  Synthetic peptide
RTVKKNIIEEN

SEQ ID NO: 20 - protein, 34 aa, Artificial Sequence; identical to SEQ ID NO: 16
  Synthetic peptide

SEQ ID NO: 21 - protein, 34 aa, Artificial Sequence
  Synthetic peptide
EASNCFAIRHFENKFAVETLISSRTVKKNIIEEN

SEQ ID NO: 22 - protein, 6 aa, Artificial Sequence
  Synthetic peptide
CIIEEN

SEQ ID NO: 23 - protein, 34 aa, Artificial Sequence
  Synthetic peptide
EASNSFAIRHFENKFAVETLICSRTVKKNIIEEN

SEQ ID NO: 24 - protein, 25 aa, Artificial Sequence
  Synthetic peptide
EASNCFAIRHFENKFAVETLICFNL

SEQ ID NO: 25 - protein, 34 aa, Artificial Sequence
  Synthetic peptide
EASNCFAIRHFENKFAVETLICFNLFLNSQEKHY

SEQ ID NO: 26 - protein, 13 aa, Artificial Sequence
  Synthetic peptide
CFNLFLNSQEKHY

SEQ ID NO: 27 - protein, 22 aa, Artificial Sequence
  Synthetic peptide
PFHSSDIQRDELARSGTGVSRE

SEQ ID NO: 28 - protein, 23 aa, Artificial Sequence
  Synthetic peptide
KEMIFNAERVGGLEERESGPRLE

SEQ ID NO: 29 - protein, 37 aa, Artificial Sequence [sequence omitted]

SEQ ID NO: 30 - protein, 23 aa, Artificial Sequence
  Synthetic peptide
SELNIYQYGGGNSALALQTDARN

SEQ ID NO: 31 - protein, 23 aa, Artificial Sequence
  Synthetic peptide
SSIDLTQRGFGNSATLDQWNGKN

SEQ ID NO: 32 - protein, 19 aa, Artificial Sequence
  Synthetic peptide
SSVNVTQVGFGNNATAHQY

SEQ ID NO: 33 - protein, 7 aa, Artificial Sequence
  Synthetic peptide
GNNQQNY